(19) United States
(12) Patent Application Publication
(10) Pub. No.: US 2023/0338480 A1
(43) Pub. Date: Oct. 26, 2023

(54) A SYSTEM TO IMPROVE HAEMOSTATIC CONTROL

(71) Applicant: Tissue-Link ApS, Ringsted (DK)

(72) Inventors: Lars Winther, Ringsted (DK); Peter Aadal Nielsen, Ringsted (DK); Kurt Baekgaard Osther, Ringsted (DK); Charlotte Videbaek, Ringsted (DK)

(21) Appl. No.: 18/245,226

(22) PCT Filed: Sep. 16, 2021
(86) PCT No.: PCT/EP2021/075533

§ 371 Date: Mar. 14, 2023

(30) Foreign Application Priority Data
Sep. 16, 2020 (EP) 20196512.6

(51) Int. Cl. A61K 38/48; A61P 7/02; A61K 47/42

(52) U.S. Cl. CPC A61K 38/4846; A61P 7/02; A61K 47/42; A61K 9/70

(57) ABSTRACT

The present invention relates to a safe and efficient system to improve haemostatic control by immobilization of Tissue Factor protein to a dressing material suitable for use as or in a wound dressing, bringing Tissue Factor in direct contact to the wound. The present invention aims at using immobilized Tissue Factor to generate a “thrombin burst”, a process by which thrombin, the most important constituent of the coagulation cascade is released very rapidly. There is no systemic release of the protein Tissue Factor into the vascular bed. The linked protein Tissue Factor can be used alone or in combination with other human proteins or components to optimise haemostasis.

### FIELD OF THE INVENTION

The present invention relates to a fast acting and safe system for promoting haemostasis while preventing undesirable systemic coagulation (e.g. disseminated intravascular coagulation). More precisely, the system comprises one or more haemostatic agent(s), including Tissue Factor (TF), immobilised on a dressing material whereby the system is capable of initiating local clot formation at the site of injury, while at the same time be safe to use.

The present invention uses immobilized Tissue Factor to generate a local “thrombin burst”, a process by which thrombin is released rapidly.

### BACKGROUND TO THE INVENTION

According to the American Association for the Surgery of Trauma, trauma is the leading cause of death for all persons between the ages of 1 and 45 years. Globally, injury is responsible for more than 5 million deaths per year. The majority of preventable deaths that occur following a traumatic event is the result of bleeding (haemorrhage), both in civilians and in military personnel (Bellamy R. F. Causes of death in conventional warfare, Mil Med. 1984; 149:55-62).

Tissue Factor (TF), also called factor III, is a transmembrane glycoprotein with an extracellular domain, which is available for interaction with extracellular components. Tissue Factor is known to initiate blood clotting when exposed to whole blood. It functions as the high-affinity receptor for factor VII and activated factor VII. The resulting complex provides a catalytic event that is responsible for initiation of the coagulation protease cascades by specific limited proteolysis (Grover S. P. and Mackman N. Tissue Factor An Essential Mediator of Hemostasis and Trigger of Thrombosis, Arterioscler Thromb Vasc Biol. 2018; 38:709-725).

Coagulation (thrombogenesis) is the process by which blood form clots, and it is an important part of haemostasis. The cessation of blood loss from a damaged blood vessel or damaged vessels, wherein damaged blood vessel(s) wall covered by a platelet and fibrin-containing clot is necessary to stop bleeding and begin repair of the damaged vessel. Disorders of coagulation can lead to an increased risk of bleeding (haemorrhage), or obstructive clotting (thrombosis).

There is general scientific consensus that there are two pathways that are able to initiate blood coagulation: The so-called extrinsic pathway and the intrinsic pathway—or often referred to as the contact pathway.

The intrinsic pathway has no known bleeding aetiology associated with it; thus, this pathway is considered accessory to haemostasis.

The extrinsic pathway is activated by an injury to the blood vessel wall, after which, Tissue Factor, also called factor III, or CD142, is constitutively expressed by cells surrounding blood vessels present in the sub-endothelial tissue. Activated intrinsic factor VIIa binds to Tissue Factor and forms the Tissue Factor-FVIIa complex, which is the key initiator of the coagulation protease cascade. The Tissue Factor-FVIIa complex assembles on a negatively charged membrane surface in a calcium dependent manner to form an enzyme complex, which proteolytically converts factor IX and X to factor IXa and Xa, respectively.

Factor IXa and factor Xa are enzymatic components of the intrinsic factor Xase and the prothrombinase complexes, respectively, which induce conversion of prothrombin (factor II) to thrombin (factor IIa). The combined activity of the activated intrinsic factors leads to an explosive burst of thrombin (FIIa). Once thrombin is generated, it cleaves fibrinogen releasing fibrinopeptides A and B—often referred to as FPA and FPB, respectively, and activates factor XIII to form a cross-linked fibrin clot.

The haemostasis has three major steps: 1) vasoconstriction, 2) temporary blockage of the endothelial defect by a platelet plug, and 3) blood coagulation, or formation of a fibrin clot, which is an integral part of haemostasis.

It is assumed that trigging of the coagulation cascade by formation of Tissue Factor-VIIa complex shall take place directly on—or directly in connection with a surface of an appropriate membrane—to express maximum proteolytic activity toward natural substrates factor IX, X, and VII (W. Ruf, A. Rehemtulla, J. H. Morrissey, and T. S. Edgington “Phospholipid-independent and -dependent interactions required for Tissue Factor receptor and cofactor function” J. Bio. Chem., 266, 2158-2166, (1991); M. M. Fiore, P. F. Neuenschwander, and J. H. Morrissey “The biochemical basis for the apparent defect of soluble mutant Tissue Factor in enhancing the proteolytic activities of factor VIIa”, J. Bio. Chem., 269 (1), 143-149, (1994)). Thus, Tissue Factor's extracellular and transmembrane domains play distinct roles in the blood coagulation process (Saulius Butenas, “Tissue Factor Structure and Function”, Scientifica (Cairo), 2012: 964862. (2012)). In addition, literature teaches that Tissue Factor proteins lacking both the cytoplasmic and transmembrane domains cannot bind to the membrane, and therefore, while forming complexes with factor VIIa, are not efficient (if active at all) in proteolyzing natural substrates factors IX, and X (W. Ruf, A. Rehemtulla, J. H. Morrissey, and T. S. Edgington “Phospholipid-independent and -dependent interactions required for Tissue Factor receptor and cofactor function” J. Bio. Chem., 266, 2158-2166, (1991); M. M. Fiore, P. F. Neuenschwander, and J. H. Morrissey “The biochemical basis for the apparent defect of soluble mutant Tissue Factor in enhancing the proteolytic activities of factor VIIa”, J. Bio. Chem., 269 (1), 143-149, (1994)).

There is no clear scientific consensus of the specific mode of action, structure-activity relationship, source of Tissue Factor, posttranslational modifications, or the role of the disulphides and glycosylation of the extracellular domain, as summarized by e.g. Butenas (Saulius Butenas, “Tissue Factor Structure and Function”, Scientifica (Cairo), 2012: 964862. (2012))

It is well-known from clinical practice that a high concentration of a haemostasis promoting compound in the circulation could potentially be fatal, as blood clots can be formed. For this reason, systemic administration of factor VII or other haemostasis enhancing agents to control bleeding can result in unwanted and fatal blood clotting at several sites including the brain and therefore these treatments are only used, if no other option is available (O'Connell K A, Wood J J, Wise R P, Lozier J N, Braun M M, JAMA. 2006 Jan. 18; 295(3):293-8).

Dressings for local treatment to control bleeding may be a convenient alternative to systemic administration. For this reason, numerous wound dressings for haemostatic control, wound healing and treatment have been developed, in response to the need for managing and protecting wounds and stopping potentially fatal bleedings.

Wound dressings are available in different shapes, sizes and materials, e.g. in layered constructs or composite materials, in the form of e.g. sheets, tampons, or suture, in addition to secondary dressings and cover dressings like wraps, gauze and tape.

A review of various wound dressing materials can be found in “Advances in Wound Healing Materials” by Willi Paul and Chandra P. Sharma (Smithers Rapra, 2015) and biological materials “Wound Healing Biomaterials—Volume 2: Functional Biomaterials” by Magnus Agren (Woodhead Publishing, 2016). Another recent review on the use of topical haemostats in trauma and emergency surgery and the various products and solutions and some safety issues can be found in Chiara et al. BMC Surgery (2018) 18:68. The same reference also summarizes the complexity and various trauma and surgery conditions in which haemostats can work.

A large number of specialized types of wound dressings have been developed, both for immediate and longer term wound management, for example: Hydrocolloid dressings, which are used on burns, light to moderately draining wounds, necrotic wounds, under compression wraps, pressure ulcers and venous ulcers. Hydrogel dressing, which is used for wounds with little to no excess fluid, painful wounds, necrotic wounds, pressure ulcers, donor sites, second degree or higher burns and infected wounds. Alginate dressings are used for moderate to high amounts of wound drainage, venous ulcers, packing wounds and pressure ulcers in stage III or IV. Collagen dressing can be used for chronic or stalled wounds, ulcers, bed sores, transplant sites, surgical wounds, second degree or higher burns and wounds with large surface areas. These types of wound dressings also all work in a passive manner to stop acute bleeding and facilitate wound healing. A historical overview of current wound dressings and technologies can be found in “Wound dressings—a review”, Biomedicine (Taipei). 2015 December; 5(4): 22.

Recently, a variety of dressings has been developed to improve the effectiveness of emergency intervention by providing an active type of dressing for use in situations of excessive or uncontrolled bleeding. These dressings contain active components like thrombin, fibrin and/or fibrinogen which have been combined with dressings or substrates which can form polymer networks, including gelatine- or polysaccharide-based dressings, glycolic acid or lactic acid-based dressings and a collagen matrix. Examples of such active dressings are disclosed in U.S. Pat. Nos. 6,762,336, 6,733,774 and PCT publication WO 2004/064878 A1.

Examples of thrombin containing products include “D-Stat Dry” from Vascular Solutions/Mallinckrodt or Thrombi-Gel from Pfizer. Examples of human fibrinogen and thrombin containing dressing include TachoSil from Nycomed/Tadeka.

The presence of human haemostatic components in these dressings, although sometimes recommended, increases the cost of the dressings and raises questions of availability of certain raw materials and risk of transmission of pathogens. In Blood Reviews 30 35-48 by Di Minno et. Al, (2016), the pathogen safety of blood/plasma-derived and recombinant products are discussed.

Examples of Tissue Factor containing products are described in the patent applications WO 2016/150449, WO 2006/047684 and WO 2012/142317.

The tissue factor and dressing combination have previously been described in detail trying to actively promote haemostasis and stop the bleeding. For example, WO 2016/150449 A1 describes various recombinant soluble human tissue factor (rshTF) molecules, especially from the HEK expression system, impregnated into a dressing, to be brought into close contact with the wound to promote haemostasis and stop the bleeding. There is no mention of any linkage of tissue factor to the dressing to prevent tissue factor being washed away by the stream of blood.

The inventor of WO 2016/150449 A1 explicitly describes the importance of having a freeze-dried tissue factor composition, which is easy to dissolve and administer to the bleeding wound. Several compositions of various sugars and other components are mentioned, all to make sure the freeze-dried tissue factor is easy to dissolve and apply to the wound. This teaches against using tissue factor permanently immobilized and covalently conjugated to a scaffold or a dressing.

The resulting product taught by WO 2016/150449 A1 would potentially create an un-safe clotting product which would be dangerous to use and not likely to be approved by authorities for treatment of human injuries.

The WO2016/150449 A1 (D1) reference describes no immobilization of rshTF to a support material, hindering the rshTF from being released and washed away from its support material.

As described previously, multiple dressings are known with various active components for enhancing haemostasis. They include dressings with impregnated active components, e.g. dressings impregnated with Kaolin (Quick-Clot, Z-Medical) or dressings impregnated with fibrin or other components which can enhance the haemostasis.

The term “impregnated” is commonly and technically understood as the method to saturate or infuse—or to fill pores or spaces in a substance. Sometimes the alternative terms lacing or coating or dried into are used to describe how the active components are incorporated in the dressing.

In technical terms, a coating is a covering that is applied to a surface, usually referred to as a substrate or scaffold. Coated systems are merely passively treating the dressing with a solution holding the active components in question, eventually followed by a drying process.

Another wound dressing system impregnated with a haemostatic agent such as tissue factor and a phospholipid system is described in WO 2012/142317 A1. The authors describe the urgent needs for a fast clotting dressing.

The same focus on efficacy and not safety is described in WO 2006/047684 A2 by Morris et al., which concerns natural or recombinant truncated Tissue Factor incorporated in soluble nanoscale particles to be used in therapeutic compositions for use in humans or animals and describes several constructs for phospholipids and scaffold protein to have the most activity in the resulting TF Nano discs.

WO 2006/047684 A2 clearly teaches away from using Tissue Factor directly conjugated to a carrier or dressing material. The application teaches us to use phospholipid-protein particle constructs to obtain a functional system for increasing blood clotting. There is no mentioning of direct linkage of tissue factor to surfaces, particles or components in the particles and they have apparently not anticipated the safety problem of washing out free tissue factor into the into the vascular bed from the particles.

Another example of a specialized type of wound dressing is described in the patent application WO 2013/007266, wherein a wound dressing matrix is described as designed to slowly release free thrombin into the wound over an extended period in a controlled manner to enhance haemostasis in the wound. Here, thrombin is distributed on the top of the polyurethane foam material by electro spraying techniques.

In addition, wound dressings can be classified as either absorbable or non-absorbable depending on whether the dressing will degrade relatively fast, alternatively be absorbed over time. Polyurethane is an example of a non-absorbable dressing, whereas e.g., oxidized cellulose or chitosan is absorbable over time.

Two frequently utilized products selected for use in battlefield casualties have been focused on chitosan-based dressings HemCon (HemCon Medical Technologies, Port-land, OR) and the zeolite granulate, zeolite beads or kaolin-coated gauze QuikClot (Z-Medica, Wallingford, CT) products used in connections with relatively minor bleedings often caused by damaged veins or superficial cuts.

None of the available dressings are based on immobilized Tissue Factor.

The safety concerns for dressings with leaking active components have been described several times, including Polish Journal of Veterinary Sciences Vol. 19, No. 2 (2016), 337-343. Despite of these concerns in the field, there has been little focus in the known dressings on the safety risks associated with leakage of Tissue Factor into the blood stream and thereby promotion of undesirable blood coagulation posing a significant risk to the patient.

To our knowledge, all prior art clearly teaches away from and does not anticipate permanent linkage of tissue factor directly to a dressing to stop severe haemorrhage, while being secure in use.

Thus, there is a need for development of new wound dressings addressing the safety concerns associated with known dressing to limit the patient's risk of undesirable side effects of treatment with the known wound dressings.

### SUMMARY OF THE INVENTION

In a first aspect, the present invention relates to a safe system for promoting rapid haemostasis while preventing undesirable blood coagulation, said system comprises Tissue Factor or any variant thereof, also referred to as Tissue Factor Component, or at least the extracellular domain thereof, and a dressing material, such as a matrix for holding the Tissue Factor or any variant thereof, wherein the Tissue Factor or any variant thereof is associated with the dressing material, whereby a linkage or interaction between the dressing material and the Tissue Factor or any variant thereof prevents the Tissue Factor or any variant thereof from dissociating from the dressing material when exposed to a physiological environment, such as an environment in which blood coagulation may be initiated.

Thus, specifically there is provided a system comprising Tissue Factor (TF) or any variant thereof, and a dressing material to which the Tissue Factor or any variant thereof is linked in such a way that the Tissue Factor or any variant thereof is prevented from dissociating from the dressing material when exposed to a physiological environment.

The system according to the present invention is suitable for promoting rapid haemostasis while preventing undesirable blood coagulation such as systemic blood coagulation and thus provides an improved remedy for treatment of severely bleeding trauma patients, e.g. trauma victims from various origin as military or terror related combat, severe accidents, and other types of injury. The system according to the present invention more specifically provides enhanced and efficient initiation of blood clotting by combining an extrinsic factor with bleeding containing extrinsic and intrinsic coagulation factors inducing endogenous thrombin burst and haemostasis, thereby providing an adequate approach to obtain efficient local control of haemostasis, especially in the context of said bleeding trauma.

Furthermore, the system according to the present invention provides a safe way to use an extrinsic factor such as Tissue Factor. Here, the extrinsic factor is linked to a dressing during the treatment of bleeding trauma patients overcoming the risk of Tissue Factor release into the blood stream, which may be the case if one on the other hand try using “free” extrinsic Tissue Factor applied to a dressing, and thus if mixed indiscriminately into a severely bleeding area, where the factor might enter into the vascular bed and may easily cause eminent risk of systemic blood clot formation in the vascular bed.

Thus, the core of the present invention is to initiate and promote haemostasis and stopping severe haemorrhage—while at the same time be safe to use, as because the tissue factor is securely linked to a dressing. The protein is permanently immobilized or bound, while still active to initiate haemostasis.

An important aspect of the present invention is the specific combination of both the efficacy and safety aspect due to low or no leakage of the active tissue factor into the blood stream, wound cavity or body.

The permanent binding of the biologically active tissue factor protein by permanent immobilization by linking is both crucial and a novel way to obtain both efficient haemostasis at the site of the bleeding, and to prevent leaked tissue factor from initiating uncontrolled haemostasis other places in the vascular system.

This aspect is especially important for severe bleedings and haemorrhage, where large amounts of blood continuously flow and easily can wash unbound tissue factor out of a dressing, while the dressing is mechanically compressed against the injury. The leakage of tissue factor can potentially initiate and cause dangerous thrombosis in the vascular system, which is an unacceptable safety risk.

This aspect of the invention is further important, as the final dressing is controlled by strict governmental regulatory guidelines, by e.g. EMA or FDA. In order to comply, both efficacy and safety is to be demonstrated.

Tissue Factor catalyses the haemostasis and does not participate in the formed network of cross-linked fibrin. Therefore, the dressing and the formed network is not covalently connected. This is in contrast to most other systems, which utilize protein components, which are included in the formed network. This is of advantage, as the dressing can more easily be removed or changed.

In the practical application on the bleeding patient, the desired blood coagulation is started by the fast forming of a mechanically stable network by applying the dressing. This is seen as a gelling of the blood and the forming of blood clots, which are sticky and have viscoelastic properties. This results in haemostasis, the ending of blood loss from a damaged vessel, followed by repair.

A system according to the present invention may further comprise Factor VII or components thereof.

A system according to the present invention may further comprise co-active agents, e.g. chitosan, salts, zeolites, kaolin, thrombin, fibrin, fibrinogen and further enhancing the efficiency of the system, as well as stabilizing agents, e.g. carbohydrates, coating proteins, pH buffers, detergents, moisture and wetting controlling agents, radical quenchers, UV adsorbers and antimicrobial agents providing prolonged storage life of the system.

In a second aspect, the present invention relates to a method of using the system of the invention for promoting rapid haemostasis while preventing undesirable systemic blood coagulation. In such a method the system is applied on the injured part of the body for a period of time allowing the bleeding to stop.

### BRIEF DESCRIPTION OF THE DRAWINGS

FIG. 1 shows the principle of the present invention, immobilization of Tissue Factor to an activated wound dressing. The single or multiple linkages anchoring the protein to the dressing and the various lengths and types of linkers. The linkers being short or elongated. The linkers being predominantly semi flexible.

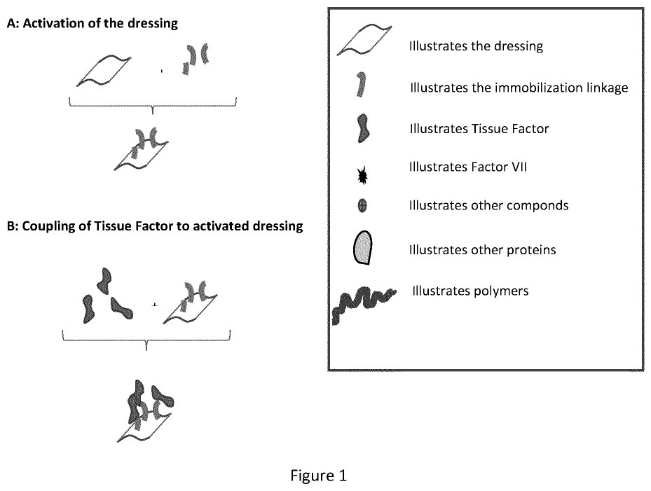

Also, the various components used in the following FIGS. 1-7 are summarized in FIG. 1.

FIG. 2 illustrates examples of the various possible ways of binding tissue factor to the dressing. (a) Through a short linker, (b) through longer linkers, and some linkers not used and quenched, (c) immobilized together with a neutral or active components, (d) through linkers which bind and crosslink tissue factor at several points, so-called multipoint attachment, (e) tissue factor immobilized to the dressing and covered with other active components, including e.g. factor VII, or (f) tissue factor immobilized to a dressing, which is absorbable. The various examples can be combined.

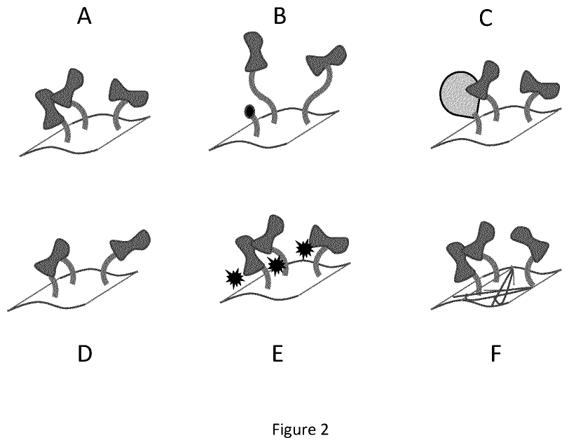

FIG. 3 illustrates examples of the various possible ways of binding tissue factor to the dressing, combining polymers and materials. (a) tissue factor immobilized to the dressing through a polymer, e.g. PEI, Chitosan or polylysine, giving longer spacer arms, (b) tissue factor immobilized to the dressing through a dendrimeric or branched linker system, (c) tissue factor immobilized to the dressing and covered with other active components, including e.g. factor VII and in a wet or dried matrix together with additional compounds (d) tissue factor immobilized to a dressing, which is combined with other dressing layers. The various examples can be combined.

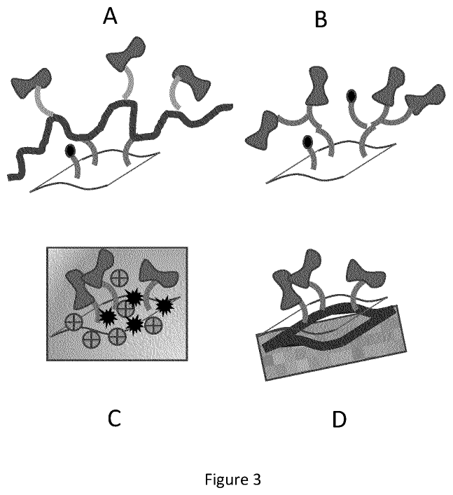

FIG. 4 shows the principle of the present invention, (a) the covalent attachment to a wound dressing of Tissue Factor and other proteins (e.g. BSA), which may have stabilizing effect(s) on Tissue Factor, (b) the covalent attachment of Tissue Factor to a suture, a linear tread.

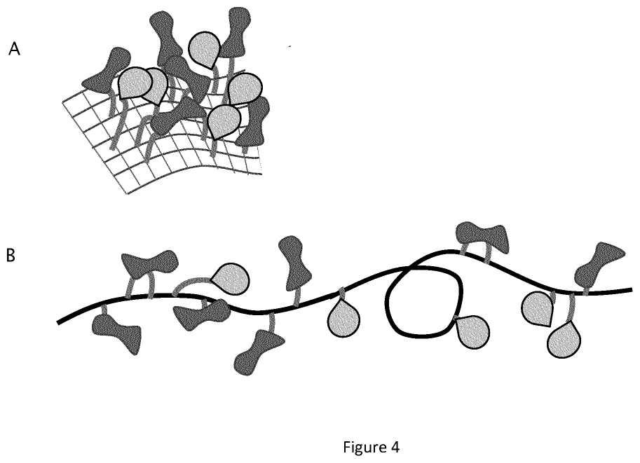

FIG. 5 illustrating the formation of cross-linked fibrin network forming the clot, catalysed by the tissue factor immobilized to the dressing.

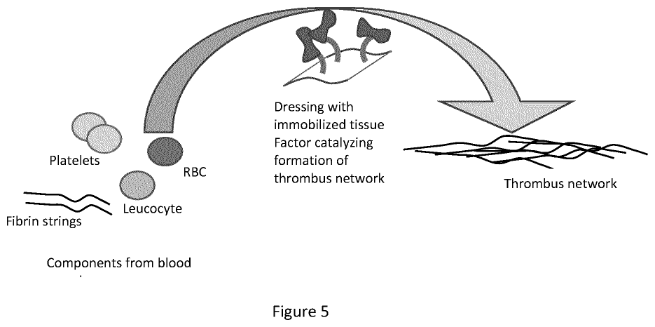

FIG. 6 illustrating the formation of cross-linked fibrin network forming the clot, catalysed by the tissue factor immobilized to the dressing and the Factor VII.

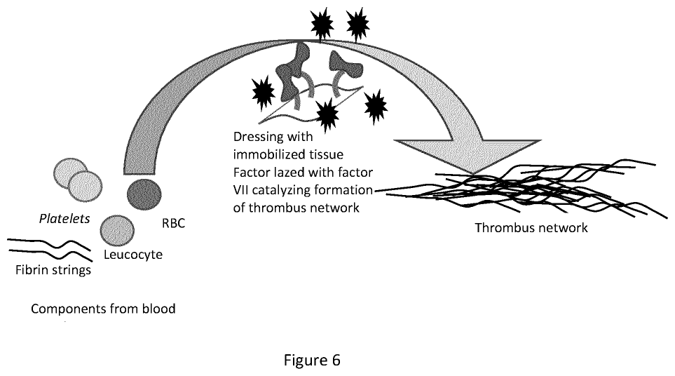

FIG. 7 shows the principle of the present invention with tissue factor immobilized to the dressing catalysing the formation of a blood clot to stop the bleeding from the injury in the blood vessel.

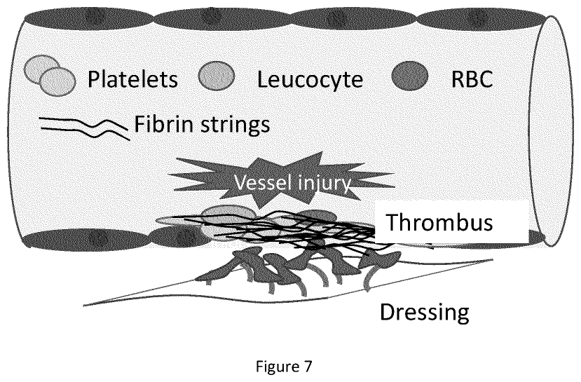

FIG. 8 illustrating the reaction scheme of divinylsulfon (DVS) activation of a hydroxylic dressing material, resulting in a vinylsulfon activated dressing. This activated dressing can react with a protein, e.g. tissue factor. At lower pH, the thiols are more likely to react first, and at higher pH, the amines will react to form a covalent bond.

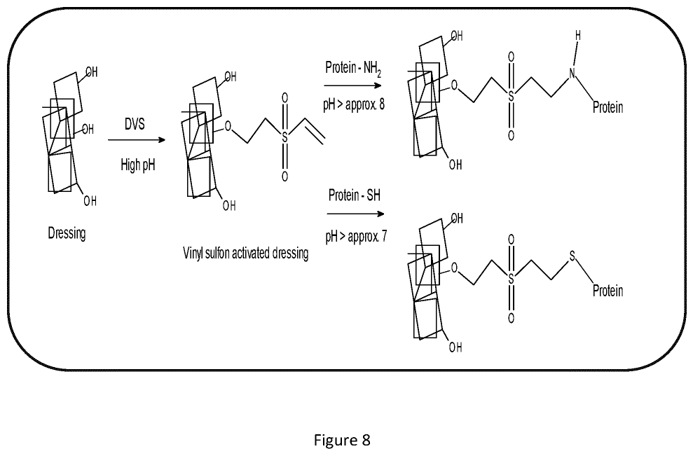

FIG. 9 illustrates the reaction scheme of epichlorohydrin activation of a hydroxylic dressing material, resulting in an reactive epoxy group activated dressing. This epichlorohydrin activated dressing can react with a protein, e.g. tissue factor. At lower pH, the thiols are more likely to react first, and at higher pH, the amines will react to form a covalent bond.

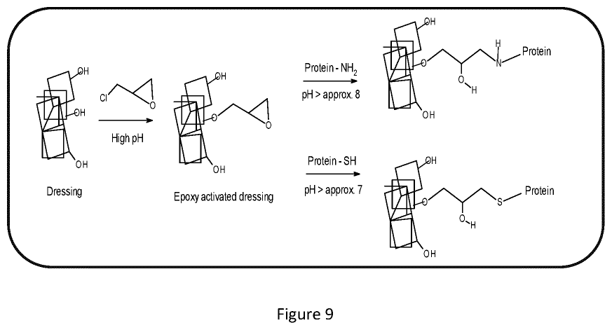

FIG. 10 shows photographs of dressing with various amounts of coupled fluorescent labelled test protein BSA as seen in example 3.

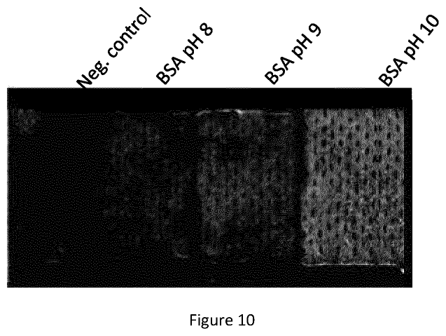

FIG. 11 shows photographs of an ex-vivo blood clotting experiment in accordance with the experimental disclosure in examples 4, 5 and 6.

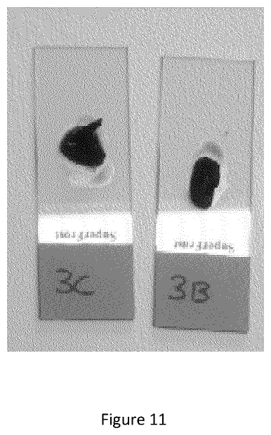

### DETAILED DESCRIPTION OF THE INVENTION

The present invention relates to a system comprising a pharmacologically active agent immobilized on a dressing material and the use thereof in a treatment consisting of efficient haemostasis of serious bleedings related to trauma in a mammal, such as a human. The system promotes rapid and sustained haemostasis.

More specifically, the system promotes rapid haemostasis initiated by a Tissue Factor immobilized on a dressing material, so that the compound is prevented from being released into the blood stream. Thereby, the compound is capable of initiating coagulation starting directly at the site of application of the Tissue Factor linked to the dressing material placed directly onto the vascular injury. This creates efficient haemostasis that at the same time is safe to use.

The invention thus allows local application of an active extrinsic agent with or without co-agents to the site where they exert their pharmacological effect by direct interaction with Factor VII/VIIa where the extrinsic agent has been prevented from entering the vascular bed of the organism and thereby hindering intra-vascular systemic exposure.

The Tissue Factor used in the present invention can be any variant of Tissue Factor as further described below and may comprise or consist of the extracellular domain. In light of what is known in relation to the function of Tissue Factor as described above, it is highly surprising that the extracellular domain without the transmembrane part of Tissue Factor can initiate the coagulation cascade and thereby actively contribute to haemostasis.

Further, it is surprising that Tissue Factor firmly (covalently) linked to the dressing material does not prevent the correct binding of Factor VII/VIIa to Tissue Factor, and thus enabling the linked Tissue Factor to initiate the clotting cascade.

Also, the inventors have found that unbound and free Factor VII together with Tissue Factor firmly (covalently) linked to the dressing material enhances the coagulation cascade, both in respect of speed and efficiency of haemostasis.

Further, the present invention is compatible with other dressing technologies aiming at solving the same problem. For example, in combination with zeolite and kaolin containing dressings, chitosan-based dressings or dressings containing fibrinogen or thrombin. Yet other examples are mentioned in the Background of the Invention Section and can be used for the present invention.

The present invention is compatible with different shapes, sizes and material types, including non-absorbable and absorbable dressing materials.

In the following, Tissue Factor, source of Tissue Factor, Factor VII, immobilization, methods of immobilization, crosslinker, linker-systems, dressings, and co-factors of relevance for the invention are described in more detail.

Definition of Tissue Factor

Tissue Factor (TF), also known as factor III or CD142, is a transmembrane glycoprotein with an extracellular domain functioning as a high-affinity receptor for the coagulation factor VII and activated factor VII (Factor VIIa). In the context of the present invention, the term “Tissue Factor” or “TF” refers to any mammalian Tissue Factor, comprising at least the extracellular domain, either endogenous or recombinant, ineluding mouse, rat, rabbit, guinea pig, dog, cat, porcine, cow and human Tissue Factor.

The human (extrinsic) Tissue Factor consists of 263 amino acids constituting the extracellular, transmembrane, and the cytoplasmic domains. The soluble Tissue Factor consisting of 219 amino acids corresponds to the extracellular domain only. The extracellular domain of human Tissue Factor has the following DNA and amino acid sequence (also shown in SEQ ID NO: 1 & 2, respectively)

```
tcaggcactacaaatactgtggcagcatataatttaacttggaaatcaactaatttcaag
S G T T N T V A A Y N L T W K S T N F K
acaattttggagtgggaacccaaacccgtcaatcaagtctacactgttcaaataagcact
  T I L E W E P K P V N Q V Y T V Q I S T
aagtcaggagattggaaaagcaaatgcttttacacaacagacacagagtgtgacctcacc
  K S G D W K S K C F Y T T D T E C D L T
gacgagattgtgaaggatgtgaagcagacgtacttggcacgggtcttctcctacccggca
  D E I V K D V K Q T Y L A R V F S Y P A
gggaatgtggagagcaccggttctgctggggagcctctgtatgagaactccccagagttc
  G N V E S T G S A G E P L Y E N S P E F
acaccttacctggagacaaacctcggacagccaacaattcagagttttgaacaggtggga
  T P Y L E T N L G Q P T I Q S F E Q V G
acaaaagtgaatgtgaccgtagaagatgaacggactttagtcagaaggaacaacactttc
  T K V N V T V E D E R T L V R R N N T F
ctaagcctccgggatgtttttggcaaggacttaatttatacactttattattggaaatct
  L S L R D V F G K D L I Y T L Y Y W K S
tcaagttcaggaaagaaaacagccaaaacaaacactaatgagtttttgattgatgtggat
  S S S G K K T A K T N T N E F L I D V D
aaaggagaaaactactgtttcagtgttcaagcagtgattccctcccgaacagttaaccgg
  K G E N Y C F S V Q A V I P S R T V N R
aagagtacagacagcccggtagagtgtatgggccaggagaaaggggaattcagagaatga
  K S T D S P V E C M G Q E K G E F R E
- taa
```

In the context of the present invention, “a variant of Tissue Factor” is to be construed as any variant able to bind Factor VII and/or Factor VIIa provided the activity of the Tissue Factor is maintained, when being linked covalently or chemically to a dressing structure to react with mammalian whole blood. Thus, the variant can be the extracellular domain of human Tissue Factor described above, or any of the other Tissue Factor molecules, molecules or parts described herein. The ability of a Tissue Factor variant to bind Factor VII may be evaluated by any suitable interaction assay such as standard pull down assays of tagged versions of the respective proteins.

The mammalian coagulation pathway is a highly conserved and robust mechanism. Applying well known alignment software, such as Standard Protein BLAST from National Center for Biotechnology Information (NCBI), it may be appreciated that the amino acid sequence (SEQ ID 2) of the extracellular domain of human Tissue Factor shares 59% identity with the extracellular domain of mouse Tissue Factor, 59% with Norwegian rat, 69% with guinea pig, 79% with cat, 76% with dog, 75% with cattle and 74% with pig. In addition to the amino acid sequence conservatism of Tissue Factor across species, it was found that recombinant soluble human Tissue Factor was capable of initiating coagulation when exposed to rat whole blood and likewise when recombinant soluble human Tissue Factor is exposed to porcine whole blood.

Thus, in the context of the present invention, “a variant of Tissue Factor” may further be defined as Tissue Factor comprising at least the extracellular domain and for which the extracellular domain shares at least 59% protein sequence identity, such as at least 69% protein sequence identity, preferably at least 75% protein sequence identity, more preferably at least 79% protein sequence identity, yet more preferably at least 85% protein sequence identity, even more preferably at least 90% protein sequence identity and most preferably at least 95% protein sequence identity with the extracellular domain of human Tissue Factor.

Motifs involved in interactions with FVIIa include aa16-aa24, aa37-aa51, aa56-aa61, aa74-aa76, aa91-aa96, aa109-aa110, aa128-aa135, aa140, aa158-aa164, aa186-aa188, aa203-aa209.

Conserved sequences across species include aa8-aa79 (AY NLTWKSTNFK TILE-WEPKPV NQVYTVQIST KSGDWKSKCF YTTDTECDLT DEIVKDVKQT YLARVFSYP) and aa91-aa214 (EPLYENSPEF TPYLETNLGQ PTIQSFEQVG TKVNVTVEDE RTLVRRNNTF LSLRDVFGKD LIYTLYYWKS SSSGKKTAKT NTNEFLIDVD KGENYCFSVQ AVIPSRTVNR KSTDSPVECM GQEK). Particular conserved sequences include aa9-aa70 and aa108-aa210 and even more conserved are the specific sequences aa17-aa19, aa34-aa39, aa52-aa68, aa125-aa129, aa132-aa137, aa159-aa167, aa184-aa187 and aa209. The highly conserved sequences are reflected in an overall large homology and identity of the extracellular domains of Tissue Factor across species.

A variant of Tissue Factor as used herein can be a molecule comprising one or more of the above-described conserved sequences or motifs.

Identity with other species:

| | Identity | |
|---|---|---|
| | (%) | Accession no. |
| Cavia Porcellus (guinea pig) | 69 | NP_001166375.1 |
| Felis catus (Cat) | 79 | XP_006934991.2 |
| Canis lupus familiaris | 76 | NP_001019811.1 |
| Bos taurus (Cattle) | 75 | NP_776303.1 |
| Sus scrofa (Porcine) | 74 | NP_998950.1 |
| Mus musculus (mouse) | 59 | AAH24886.1/NP_034301.3 |
| Rattus norvegicus (rat) | 59 | NP_037189.2 |

Source of Tissue Factor

Tissue Factor suitable for use in the context of the present invention may be obtained from mammalian whole blood, preferably from human whole blood or by standard recombinant technology.

Standard industrial recombinant technology including use of human embryonic kidney HEK, Human 293/T, HEK-F, hamster BHK 21, Chinese Hamster ovarian cells (CHO), SF9 insect, mouse, E. coli, or yeast (Saccharomyces cerevisiae) cell systems.

Posttranslational modification performed in mammalian cells, including human cells influences several physical and chemical properties, such as glycosylation, phosphorylation, carboxylation, palmitoylation, and neuramino-glycosylation etc., which often is of significant importance for the properties of the coagulation factor.

The use a human cell line such as HEK293ts cells is preferred due to the human glycosylation. The glycosylation of recombinant proteins in general, especially those destined for potential administration to human subjects, is of much larger critical importance than previously believed. Glycosylation profoundly affects biological activity, function, clearance from circulation, and crucially, antigenicity.

A Brooks Review (Brooks, S A, Molecular Biotechnology, (2004) Vol 28, 241-256(16)) gives a brief overview of human N- and O-linked protein glycosylation, summarizes what is known of the glycosylation potential of the cells of nonhuman species, and presents the implications for the biotechnology industry.

HEK293ts cells are further preferred because the cell line is capable of being cultured in serum free medium as a suspension cell culture, the cell culture may be expanded to a cell concentration of over 3 million cells/ml or even under optimal condition to a cell culture concentration of 10 million cells/ml, and even higher when run under extremely constant physiological pH levels, such as between pH 6.8-7.4 with the optimal oxygenation and supply of necessary gasses and pressure in the vessel, in which these cells are cultured such as High cell density perfusion process in single use bioreactor systems (GE-Wave) culturing with disposable cell culture bags to up to 100 liter per GE or comparable Bioreactor Wave bag, using EX-CELL™293.

In one preferred embodiment, recombinant human Tissue Factor is obtained by ex-pressing Tissue Factor having the sequence SEQ ID 1 in HEK293ts cells.

Any suitable expression vector can be used for expression of Tissue Factor, e.g. pST2 vector for cloning and pST2-HF3 after cloning.

The Tissue Factor, including a variant or the extracellular domain, can also be synthesized by synthetic methods, or by combining recombinant methods followed by chemical modifications. The synthesis methods of stepwise elongating peptides by stepwise coupling protected amino acids or other multifunctional building blocks together is well-described from for example classical Merrifield solid phase peptide synthesis (SPPS) or in solution. The various chemo selective and orthogonal protection groups include Boc, Fmoc, Benzyl or t-But among others. The amide formation is done using for example carbodiimides, efficient active ester, HATU/HOAt or similar reactions. A summary of SPPS reagents and methods can be found in for example Merrifield, R. B. In Peptides: “Synthesis, Structures and Applications”; (Gutte, B., Ed.); Academic Press: San Diego, C A, 1995; pp 93-168 or in Fernando Albericio (ed.): “Solid-Phase Synthesis: A Practical Guide”, CRC Press 2000.

### Definition of Factor VII

Factor VII, blood-coagulation factor VIIa, activated blood coagulation factor VII, also known as proconvertin (EC 3.4.21.21) is a 50-kDa, vitamin K-dependent zymogen synthesized by the liver that is critical for initiation of tissue factor-induced coagulation (extrinsic pathway). In the context of the present invention, Factor VII includes the activated form of Factor VII, often referred to as Factor VIIa. Including the recombinant human factor VIIa (eptacog alfa, NovoSeven, Novo Nordisk, Denmark) developed treatment of uncontrolled bleeding in hemophilia patients, and the biosimilar form of recombinant activated factor VII (AryoSeven, AryoGen, Iran)

Immobilization

A system according to the present invention comprises Tissue Factor, preferably the extracellular domain, or any variant thereof immobilized on a dressing material.

The term “immobilized” and “linked” is used interchangeably and is in the context of the present invention to be understood as a molecular binding of Tissue Factor to a dressing material having a strength sufficiently high to prevent Tissue Factor from being released from the dressing material under physiological conditions, e.g. when the system is exposed to mammalian whole blood. This may be achieved by providing a molecular binding linked between Tissue Factor and a dressing material having a binding strength sufficiently high to prevent significant measurable leakage of Tissue Factor when measured by e.g. following fluorescent label techniques. Preferably, the binding strength is at least a factor 1/100000 of the reference binding strength between biotin and streptavidine as measured by e.g. fluorescent or radioactive label techniques. The dissociation constant for streptavidin and biotin is approx. 10⁻¹⁵ mol/L.

The molecular binding applied to achieve immobilization of Tissue Factor to a dressing material preferably has a dissociation constant (Kd) of at least 10⁻¹⁰ mol/L, more preferably at least 10⁻¹² mol/L. A preferred example of such a molecular binding is a covalent binding.

The term covalent or near covalent binding is a practically irreversible binding of the active compound to the dressing material through preferable stable chemical covalent bonds at physiological conditions.

The preferred covalent bonds are C—C, C—O, C—N, C—S or Si—O chemical bonds and combinations hereof, which have a bond dissociation energy (mean bond dissociation enthalpy) of more than 10 kcal/mol, preferably more than 30 kcal/mol and even more preferably more than 75 kcal/mol, resulting in stable bonds with little or no risk of release of the active component into the wound.

Methods of Immobilization

The regio and chemo selective coupling methods by combination of specific peptide sequences and reagents and without protection groups can guide the exact coupling site and preserve the peptide functionality. Several methods are known, including various “ligation” methods. An early overview can be found in e.g. “Methods and strategies of peptide ligation” by James Tam et. al. in Biopolymers. 2001; 60(3): 194-205 and Protein Synthesis “Chemoselective Ligation and Modification Strategies for Peptides and Proteins” Christian P. R. Hackenberger and Dirk Schwarzer in Angew. Chem. Int. Ed. 2008, 47, 10030-10074.

The various ligation methods useful for post-translational modifications and coupling to a dressing material include Staudinger ligation, azide-alkyne click reactions, and metal mediated metathesis reactions.

Some ligation methods, or sometimes referred to as ‘bioorthogonal chemistry’. are chemo selective towards e.g. cysteine, lysine or tyrosine. Further some ligation techniques are compatible with glycosylated proteins and used in complex environments.

A general review covering the state of art is found in e.g. Advances in Chemical Protein Modification by Boutureira and Bernardes in Chem. Rev. 2015, 115, 5, 2174-2195 and in numerous references, including in by Spears and Fascione, Site-selective incorporation and ligation of protein aldehydes”, in Org. Biomol. Chem., 2016, 14, 7622.

Of particular relevance is tissue factor expressed from CHO or HEK cells with end functionality, like Gly-His or Lys-His tags, ready for chemo selective coupling by e.g. selective amin acylation with e.g. p-methoxy phenyl ester to the an activated dressing material. Gly-His tags are described in by Martos-Maldonado et. al. in “Selective N-terminal acylation of peptides and proteins with a Gly-His tag sequence”, in Nature Communications volume 9, Article number: 3307 (2018)

Alternative molecular binding mechanisms suitable in the context of the present invention are ionic or non-covalent bonds, provided they result in a dissociation constant between the dressing material and Tissue Factor of more than 10⁻¹⁰ mol/L, more preferably at least 10-12 mol/L.

Other preferable bindings include biotin-streptavidin, lectine pairs, DNA-DNA, DNA-PNA, DNA-LNA, metal-chelates or other ligand pairs, which result in near covalent binding strengths.

Crosslinkers & Linker Systems

Immobilization of Tissue Factor to a dressing material may be achieved by a direct molecular binding using a crosslinker between Tissue Factor and a dressing material or by indirect binding through a linker or linker system.

The use of crosslinkers and linkers in conjugation chemistry to immobilize proteins to substrates including affinity chromatography columns media, membranes and diagnostic devices is technically mature, with a large available source of literature and empiric knowledge. Numerous general references are available, including the review books “BioConjugation Techniques” (Elsevier 2013, 3. ed), “Immobilized affinity ligand techniques” by Greg T Hermanson, A. Krishna Mallia, Paul K Smith (Academic Press, 1992), “Chemistry of Protein Conjugation and Cross-Linking” by Shan S. Wong (CRC Press 1991) and “BioConjugation Techniques—Strategies and Methods by Sonny S. Mark (Springer, 2011).

The linker which anchors Tissue Factor to the dressing material should preferably be semi flexible in water at physiological pH to allow a stretched configuration and thereby higher efficiency for initiating haemostasis.

In the present context, the length of the linker is defined by the number of bonds, wherein bonds are C—C, C—N, C—S, C—C(O) and/or C—O bonds. The linker preferred in accordance with the present invention comprises amide, carbamide, sulfones, ethers and sterically hindered groups, which prevent the linker from being fully flexible.

The linker can be traditional linear or of the crosslinked and branched types. Examples of these include linear PEG linkers or acrylamide based branched linkers.

Furthermore, Tissue Factor may be immobilized to a dressing material individually or in clusters on a high density dendrimer system. Examples of multibranched or dendrimer linker systems include different generations of Polyamidoamine (PAMAM) dendrimers with different surface functional groups which can be conjugated to Tissue Factor (e.g. available from Merck). Other well-known systems from diagnostic and pharmaceutical applications include polylysine, polyethyleneimine and polyester-based dendrimers and dendrons.

Instead of chemically introducing linkers, they can also be introduced directly into the recombinant protein to ease the regioselectivity of the immobilization and to create polymeric linkers with well-known molecular weight and properties. An example of this type of ligation technique is described by Novo Nordisk by Thomas Kjeldsen et. al, “Dually Reactive Long Recombinant Linkers for Bioconjugations as an Alternative to PEG” in ACS Omega 2020, July 2020.

Clusters of Tissue Factor may also be achieved by using a recombinant repeated Tissue Factor protein comprising two or more repeats of Tissue Factor, e.g. comprising two or more repeats of SEQ ID 2 in a single protein.

The inventors have found that the extracellular domain of mammalian Tissue Factor comprises numerous lysines, with primary amines in the side chain, which can be targeted chemically by chemo selective conjugation. Endogenous Tissue Factor contains 16 potential conjugatable lysines in position: 20, 28, 41, 46, 48, 65, 68, 122, 149, 159, 165, 166, 169, 181, 201, 214.

In addition, the inventors have recognized that the expected important glycosylation sites in position 11, 124, 137—especially 124 and 137—are located away from the lysines suitable for conjugation. Therefore, conjugation targeting lysines are an appropriate way of preserving the functionality of Tissue Factor i.e. to preserve its ability to bind factor VII and thereby initiate the coagulation cascade.

Furthermore, the inventors have identified the three cysteines located in position 57, 186 and 209 as potential targets suitable for regio and chemo selective conjugation.

Immobilization of Tissue Factor to a dressing material is preferably achieved by activating hydroxyl groups on the dressing material and couple these to Tissue Factor through its amino and thiol groups or introducing a further linker between dressing and the Tissue Factor.

It should be understood that numerous conjugation methods are suitable for immobilizing Tissue Factor to a dressing material, including the use of epoxides, vinylsulfones, azlactones, cyanobromide, N-Hydroxysuccinimide esters, nitrophenyl esters and other active esters, aryl halides, isothiocyanates, aldehydes, maleimide, disulphides, cyclic lactones, triazines or benzoequinone.

The preferred chemo selective conjugation method for targeting lysines is using ring-opening of epoxides from epichlorohydrin or Michael addition from divinylsulfon. These methods are generally known to couple mainly through primary amines or thiols, resulting in stable covalent links in high yields.

Epichlorohydrin is a versatile chemical intermediate, general crosslinker and organic synthon used in a variety of applications, used in e.g. the manufacture of elastomers, cross-linked food starch, surfactants, plasticizers, dyestuffs and adhesives, useful for e.g. introducing reactive epoxy groups to e.g. hydrozylic surfaces. It is described in e.g. Greg T. Hermanson G T. Bioconjugate techniques. Elsevier; Rockford, Il: 2008.) or a specific example to activate a hydroxylic surface, introduce a second linker and couple lectins Concanavalin A and Wheat Germ Agglutinin with glutaricdialdehyde by Aniulyte Jolita et. al.: “Activation of cellulose-based carriers with pentaethylenehexamine”, Proc. Estonian Acad. Sci. Chem, 1 Jan. 2006 (2006-01-01), pages 61-69.

Divinylsulfon (DVS) is a more unusual reagent, also listed among numerous reagents in the Hermanson book, and its properties further listed in JULIA MORALES-SANFRUTOS ET AL: “Vinyl sulfone: a versatile function for simple bioconjugation and immobilization”, ORGANIC & BIOMOLECULAR CHEMISTRY, vol. 8, no. 3, 1 Jan. 2010 (2010 Jan. 1), page 667.

DVS has been used for various applications, including DNA crosslinking, crosslinker in polymer networks and to immobilize the enzyme Candida antarctica lipase B using divinyl sulfone activated chitosan in order to develop a tool for organic synthesis of polyesters. As described by Pinheiro et al, ACTA PAEDIATRICA. SUPPLEMENT, vol. 130, pages 798-809.

It should be understood that the selection of conjugation chemistry and methods is not trivial; especially not when considering the sensitive nature of the Tissue Factor molecule, and the risk of losing the activity by uncontrolled multipoint attachment and crosslinking the many amino groups and thiols. Search of cross linkers will produce 1000's of references with many combinations and applications, but none concerning tissue factor, blood clots or haemorrhage.

The crosslinking reagents, epichlorohydrin or DVS, are preferred to form a linkage between all dressings having hydroxy or amino groups and Tissue Factor. First, the dressing is activated with epichlorohydrin or DVS to produce an activated dressing, which can be coupled to Tissue Factor through the epoxid or vinylsulfon functionalities.

DVS activation of hydroxyl groups at basic conditions on the dressing results in reactive vinylsulfon groups. The vinylsulfon activated dressing can be stored in water.

The vinylsulfon reacts slowly with primarily amines in the Tissue Factor protein, at slightly basic pH conditions. Any potential radical-initiated side reactions can be reduced by adding a radical quencher. This method gives a stable and short linker between dressing and protein. Thiols from cysteine react rapidly with vinylsulfon at lower pH. This allows partly control of the Michael addition chemo and regio selectivity by adjusting the pH during coupling between the dressing and the protein. (Porath, J., Laas, T., and Jansson, J.-C. (1975) Agar derivatives for chromatography, electrophoresis and gel-bound enzymes. III Rigid agarose gels, cross linked with divinyl sulfone (DVS). J. Chromatogr. 103, 49-62),

Another preferred method is the use of epichlorohydrin, which introduces an epoxide on the dressing. This functional moiety will react with amine and thiols, depending on the pH during conjugation.

Further, the dressing activated either by using DVS or epichlorhydrin can be stored before the immobilization of the Tissue Factor.

By adjusting the pH below 9 during the protein conjugation, the guanidino group in arginines in position 74, 131, 135, 136, 144 of the Tissue Factor will be less targeted, as they will be more protonated than the lysines, due to the difference in pKa and the conjugation site can thereby be limited to lysine or cysteine residues.

It was surprisingly found that a minimum component comprising only the extracellular domain of Tissue Factor can be immobilized to a dressing material and still maintain its functionality being able to initiate a natural coagulation cascade without systemic exposure and hereby be used as local treatment of e.g. bleeding wounds.

An appropriate linker technology is known to the skilled person but may advantageously include DVS and/or epichlorohydrin system(s) since both allow Tissue Factor to preserve functionality. Furthermore, both systems provide a suitable degree of activation of the dressing material, followed by the degree of loading and binding of the active protein. Finally, both systems are readily available at a low cost and provide good reproducibility. The most preferred epichlorohydrin system provides a short and semi-stiff linker containing a hydrophilic secondary alcohol moiety and no charges.

In the context of the present invention, it should be understood that one can alternatively activate the protein first and couple to the dressing or introduce various linkers between the dressing and the protein.

### Definition of Dressing

A dressing material is to be construed as any material on which Tissue Factor may be immobilized and which will not itself enter blood circulation via a bleeding wound, such as a bleeding trauma. The dressing material may be in any suitable form, e.g. a polymer, beads, a matrix, a solid carrier, a patch, a bandage, a gauze, a membrane, a barrier, a compress, a scaffold or a suture material. Thus, in general the dressing material can be a solid material, which comes in numerous physical forms including sheets, nets, woven and non-woven sheets, sutures, threads, tampons, foams and balls.

The dressing material may in itself constitute a topical dressing for local management of bleeding wounds, such as lacerations, cuts and abrasions or be integrated into such a topical dressing. Preferably, such a dressing has enough physical integrity to be pushed into or wrapped around the wound or lacerations, cuts and abrasions. The dressing will be in direct contact with the wound or blood or fluids from there. Often, the dressing is held in place by a bandage, or a compressing bandage.

A dressing comprising the system according to the present invention constitutes a functionalized dressing and can be designed to have a large surface area for adsorbing fluid from the wound or to optimize the contact area to enhance the formation of blood clot.

Suitable dressing materials include cotton, cellulose, cellulose ethers, regenerated cellulose, oxidized cellulose, carboxymethylcellulose, polyurethane, agarose or paper-based membranes, polyamides, poly sulfone ether, polyvinyl alcohols, nitrocellulose, nitrocellulose mixed esters, nylons, polycarbonate, polysulfone, polyethylene terephthalate, polyvinylidene fluoride or polypropylene, polyethylene and co-block polymers, blends and combinations hereof.

This includes materials, which contain natural or artificial polymers, which are known to catalyse and enhance haemostasis, including carbohydrates, even more specifically chitosan and chitosan derivatives, dressings which contain microfibrillar collagen or thrombin, poly-N-acetyl glucosamine, polyprolate acetate, D-glucosamine or calcium alginate, and dressings which contain inorganic or other salts known to catalyse and enhance haemostasis, including calcium chloride or other salts.

Dressing materials are classified as either absorbable or non-absorbable depending on whether the body will naturally degrade and absorb the material over time. In the context of the present invention, a suitable absorbable material will maintain its structural integrity when in contact with mammalian plasma for at least 15 min, such as 20 min, preferably up to days and not more than 200 days.

Absorbable dressing materials include oxidised cellulose, silk, catgut, synthetics polyglycolic acid, polylactic acid, polydioxanone, and caprolactone. Including polymer and co-block-polymers based on one or more of five cyclic monomers: glycolide, 1-lactide, p-dioxanone, trimethylene carbonate or ε-caprolactone.

Absorbable dressing materials are particularly preferred for providing sutures. The suture thread may be mono or multi filament suture and pre-condition-coated with another material such as e.g. a collagen coat.

A wound dressing comprising a system according to the present invention may be in a flexible or mechanically unstable form, e.g. a glue, a powder, a spray, a gel, a granulate, a mechanically compressed sponge or granulates, a swellable sponge, a gelatine, a paste, a fluid, or a cream to be applied in direct contact with the wound. Tissue Factor is immobilized on a dressing material, preferably covalently bound to the dressing material, which may be a component of the dressing or integrated therein. The immobilization prevents systemic administration of Tissue Factor.

The preferred dressing material is based on cotton, cellulose or oxidized cellulose. Factor VII

A system according to the present invention may further comprise Factor VII.

The Factor VII is preferably covered in the dressing or sprayed to the dressing immediately before use.

By applying Factor VII locally at the bleeding site, the systemic exposure is reduced. It is advantageous to use an order of magnitude less amount of Factor VII than would be used in a systemic treatment. Thereby, the safety concerns can be reduced significantly.

The preferred combination of bound Tissue Factor according to the present invention and Factor VII is by using less than 1/10 the amount at the bleeding site than would normally be used in a systemic treatment.

Co-Active Agent

A system according to the present invention may further comprise a co-active agent.

Examples of co-active agent include various agents, which further enhance haemostasis and other agents, which can benefit a patient having a bleeding wound, such as agents that can cool down the wound or reduce the apparent pain.

Preferably, such co-active agent is a haemostasis enhancing agent such as an inorganic salt, gelatine or gelatine derivatives, biopolymers like cyclodextrin or chitosan or combinations hereof. Collagen or thrombin, poly-N-acetyl glucosamine, polyprolate acetate, D-glucosamine, calcium alginate, kaolin, salts known to catalyse and enhance haemostasis, including calcium chloride, or other salts.

A co-active agent may be immobilized on the dressing material together with Tissue Factor.

A system according to the present invention may further comprise a stabilizing agent. Suitable stabilizing agents include proteins, which may be irrelevant for the haemostatic activity of the active compound. The preferred stabilizing agent will passively fill in the surface space of the dressing material around the active compound. Preferred stabilizing agents include serum albumin, fish gelatine, casein or protein mixtures or proteolytic degraded gelatine or other proteins.

The stabilizing agent may be immobilized on the dressing material together with Tissue Factor, whereby release to the vascular system and systemic exposure is minimized.

The system according to the present invention may be covered or swelled with chemicals and buffers, which will further stabilize the functionality upon storage and transport. This includes antimicrobial agents or buffers to stabilize the pH or agents, which protect against damage from freezing or UV irradiation.

The inventors have surprisingly found that dressing with immobilized linker and immobilized Tissue Factor preserved its functionality for initiating haemostasis upon prolonged storage, even without being coated (“glazing”) with a mixture consisting of carbohydrates, protein, alcohols and preservatives.

Preferred carbohydrates for further storage stabilization include trehalose, lactitol, inositol, glucose, sucrose, mannose, saccharose, dextran, diethylaminoethyl (DEAE)-dextran and/or agarose.

Preferred proteins include human serum albumin, bovine serum albumin (BSA), recombinant serum albumins and derivatives, Non-fat dry milk, Casein or caseinate, Fish Gelatine. Preferred alcohols include glycerol, and preservatives include 2-chloroacetamide or other antimicrobial agents suited for wound dressings or topical use.

A system according to the present invention provides Tissue Factor or any variant hereof in manner that resembles the manner the body normally would stop the bleeding, will allow haemostatic control, instead of relying on the smaller amount of endogenous exocrine Tissue Factor that can connect with Factor VII/VIIa at the bleeding site, without liberation of the Tissue Factor into the blood stream through the bleeding vessels.

Notably, by using sutures, powder, granulates, gels, foams, sprays or sponges, access to hard-to-reach bleedings may be enhanced.

The system of the invention can e.g. be in the form of a gel to be applied to the wound or in the wound cavity, either by hand or by a device. The advantage is the flexibility in getting to the bleeding site and the possibility to combine with any type of dressings or devices, to further put a pressure to the wound. The gel can be distributed directly to the wound or indirectly to the nearest available dressing. This is important in situations, where one may not have the correct size or type of dressing for the situation, for example during military operations at night or at civilian accidents outside the hospital.

A foam is a variation of the gel, which can be expandable due to e.g. its gas content. A further variation is sprayed foam with absorbable polymers with covalent attached Tissue Factor. When sprayed to the wound or dressing, after the neutral solvent or gas is evaporated, the remaining gel will be close to the bleeding site and concentrated. Again, to increase the flexibility to apply to the bleeding site or indirectly to dressings.

Yet another variation is the use of sponges, including compressible sponges. The sponges in the form of small beads can be delivered into the wound cavity by hand or by a device and after mechanically expansion or after swelling put further pressure on the bleeding sites.

Another variation is in the form of a sticky glue, typically absorbable, which can be applied manually or from a spray or tube device to hold tissue together and at the same time initiate haemostasis.

Rapid Haemostasis

Static whole blood spontaneously coagulates after on average 10-30 minutes, very much depending on the human it is taken from. There is a huge variation, depending on e.g. gender, age, race, general physical condition, diseases or medication taken, e.g. anticoagulants.

Therefore, a measure of the efficiency of an active haemostatic dressing can best be described as a reduction in time compared to a passive dressing or no dressing—or how long time is necessary to hold the dressing on the bleeding site.

A reduction of time to haemostasis in minutes is considered rapid haemostasis and of value. Even one minute reduction is of practical value and two or more minutes are significant improvements. But often, the effect can be the fundamental question of stopping or not stopping the bleeding after applying pressure to the dressing covering the bleed site.

Undesirable Blood Coagulation:

Any haemostasis or blood coagulation initiated by the dressing in the vascular system or organs away from the bleeding site is uncontrolled and undesirable.

Any dressing initiating or inducing haemostasis away from the bleeding site can cause blood clots, and small blood vessels in critical places can become clogged with clots. Clogged vessels in the brain can cause strokes and clogged vessels leading to the heart can cause heart attacks. Pieces of clots from veins in the legs, pelvis, or abdomen can travel through the bloodstream to the lungs and block major arteries, giving pulmonary embolism. It is mandatory to prevent undesirable blood coagulation in order have a safe haemostatic dressing.

In the present application's experimental examples, the dressings are extensively washed with various buffers after the conjugation steps. We have illustrated that the tissue factor could not be washed away, as the washed dressings with linked tissue factor could be used for promoting rapid haemostasis in an animal model with severe haemorrhage.

How to Measure Effect Ex-Vivo

The effect of a dressing to introduce haemostasis is of course measurable in human experiments and in some animal models, as described in example 8.

In example 10, we describe a simple assay, where various dressings are mixed with whole blood, a mixture of thrombocytes and plasma proteins or pure plasma in a vial.

By only using plasma from a pool of donors, the initial haemostasis process is measured and presumably donor variations reduced. On the other hand, the assay will not indicate the thrombin burst effect.

In the assay the vial is heated to 37° C. and turned over at specific time intervals. The haemostasis is measured as the time until the formed gel or clot is not running down when the vial is turned upside down. The efficiency of the dressing to initiate haemostasis is estimated as the reduction in time as compared with no dressing, untreated dressing or dressings with dummy protein.

A Thromboelastographic (TEG) viscoelastic haemostatic assay can be used to measure the total viscoelastic response of clot formation initiated by free solution tissue factor. Using e.g. whole blood, a mixture of thrombocytes and plasma proteins or pure plasma.

### Embodiments

As outlined above in one embodiment, a system according to the present invention comprising Tissue Factor or any variant thereof immobilized on a dressing material, e.g. in the form of a suitable dressing, may be brought into contact with a bleeding trauma whereby the system provides the profusely bleeding area with Tissue Factor, which interacts with Factor VII/VIIa. This will happen in significant amounts that sur-pass any other endogenous cell bound Tissue Factor, e.g. from the endothelial tissue or leukocyte, which normally can activate coagulation cascade originated from smaller bleedings, meaning bleeding that normally can be stopped by short time compression.

However, as profuse bleedings start to exceed around 100 cc of sieving blood (from smaller venous bleedings, meaning if a laceration include larger vessels and especially arterial vessels), large amount of Tissue Factor is likely in a far superior manner to prevent further profuse bleedings as long as the Tissue Factor or parts thereof is applied.

In one embodiment, Tissue Factor is immobilized to a dressing material by a covalent binding either directly or via a linker. Thereby, Tissue Factor will not be released from the dressing material in any clinically significant amount inside the vessels, at the site of vascular injury. Thus, exposure in the blood stream in a clinically relevant amount, which may cause a thrombosis or an embolic effect in the blood stream, is avoided.

In one embodiment, a wound dressing comprising a system according to the present invention is provided, more specifically the wound dressing may comprise the extracellular domain of human Tissue Factor or any variant thereof made recombinant, i.e. recombinant soluble human Tissue Factor, immobilized covalently to a dressing material forming or incorporated into a dressing forming the wound dressing. The wound dressing prevents as intra-vascular thrombosis caused by externally applied Tissue Factor. When applying the dressing with immobilized Tissue Factor to a bleeding area, it is, depending on how profuse the bleeding is, assumed that Tissue Factor interacts with sufficient amount of Factor VII/VIIa, which activates the clotting cascade. It is assumed that sufficient coagulation factors are available and therefore haemostasis can be effectively established to delay or stop the bleeding at the bleeding site(s) if there are sufficient available clotting factors to succeed in adequate haemostasis.

In yet another embodiment, a system according to the present invention combines the immobilized tissue factor and unbound Factor VII in the dressing to further speed up the haemostasis in the bleeding area.

In one embodiment, a system according to the present invention is provided in the form of a suture. A suture typically consists of a needle with an attached length of thread. All sutures are classified as either absorbable or non-absorbable depending on whether the body will naturally degrade and absorb the suture material over time. An additional preferred embodiment defines the dressings as being absorbable or, alternatively, non-absorbable

A preferred embodiment provides a system according to the present invention in the form of a sheet, threads, fibre or bundle of threads. The threads or bundle of threads can then be woven, glued or otherwise integrated into various forms of wound dressing. This allows for centralized and standardized manufacturing and easy implementation into the many different wound dressings using existing manufacturing techniques. The density of active sheet, threads, fibre or bundle is optimized for the particular application.

The inventors have surprisingly found in an experiment that a local concentration of Tissue Factor triggers haemostasis very rapidly secondary to local sustained thrombin generation. This step leads to the current invention.

### EXAMPLES

The examples illustrate the invention by disclosing the epichlorohydrin and DVS activation of dressings, optimizing the coupling using a test and space filling protein and tissue factor, an ex-vivo functional test of the dressing, test with dressing covered with factor VII solution, large scale preparation and two different bleeding performance tests in mini pigs.

In the following, a DVS and epichlorohydrin activation of dressings is disclosed.

### Example 1—DVS Activation of Dressing

Three types of dressing, A woven gaze, B dental dressing, and C densely woven cotton sheets, were washed with buffer (50 mM Carbonate, pH 11.6) and treated for 1 or 4 hours with 5% DVS in 50 mM Carbonate, pH 11.6 in a closed container while being gently stirred. The DVS solution was decanted and discarded. The dressings washed 7 times with deionized water, before being stored in deionized water added approximately 50 ppm hydroquinone.

### Example 2—Epichlorohydrin Activation of Dressing

The three types of dressing above were washed with buffer (2 N NaOH, 1:1 water-DMSO) and treated for 1 or 4 hours with 5% Epichlorohydrin (Merck) in 2 N NaOH, 1:1 water-DMSO in a closed container while being gently stirred.

The solution was decanted, collected and discarded. The dressings washed 7 times with deionized water, before being stored in deionized water. The dressings were cut in smaller pieces.

In the following, the coupling of fluorescence labelled BSA test protein to DVS or Epichlorohydrin activated dressing is disclosed.

### Example 3—Coupling of Fluorescence Labelled BSA Test Protein

The test protein, Bovine Serum Albumine—BSA, is relevant as a model, as this is useful as filler or spacer in the dressing material and is a component in commercially available innovin.

BSA was fluorescence labelled by dropwise adding 0.525 ml of a 5/6-carboxy fluorescein NHS ester (Thermo Fisher) 2 mg/ml DMF (Merck) solution to a stirred 10 ml 1.00% solution of BSA (Merck) in 0.20 M HEPES (Merck) at pH 7. After one hour stirring at room temperature, the solution was used without further purification.

A similar solution without BSA was prepared as a control.

Fluorescein labelled BSA was coupled to the DVS or Epichlorohydrin activated dressings. Different types of dressing were tested.

Different dressings at various pHs and time, with 1.0 mg/ml fluorescein labelled BSA concentration according to the general procedure at room temperature (21° C.): First washing the dressing (approx. 2×2 cm) in the reaction buffer, that is 0.10 M carbonate pH 10.0, 0.10 M Carbonate pH 9.0 or 0.10 M HEPES pH 8.0, respectively.

The dressing was shaken and dried briefly on a paper towel, before being immersed into the fluorescein labelled BSA reaction solution or control solution. The various reaction mixtures were prepared by adding water, pH buffer and fluorescein labelled BSA solution.

After reaction, any remaining active groups were quenched with a stop buffer, (0.20 M ethanolamine, 0.20 M carbonate, pH 9.0) for 15 minutes. (Similar to standard quenching procedure for epoxy activated affinity chromatography resins). The dressings were washed extensively by shaking in deionized water four times. The dressings were stored in 10 mM HEPES at pH 7.0.

The wet dressings were placed on a standard glass microscope glass before the fluorescent intensity was measured in a fluorescent slide scanner. Fluorescence images were recorded and the intensities compared, using an ImageXpress-Pico with CellReporterXpress software (Molecular Devices), with a microscope slide scanning frame.

In summary, the following was observed:

| | Dressing | | Activation | Coupling | Coupling | Fluorescent |
|---|---|---|---|---|---|---|
| No. | type | | time, hours | pH | time, hours | intensity |
| 1 | A | DVS | 4 | 9 | Negative | 0 |
| | | | | | control | |
| 2 | A | DVS | 4 | 8 | 3 | + |
| 3 | A | DVS | 4 | 9 | 3 | ++ |
| 4 | A | DVS | 4 | 10 | 3 | +++ |
| 7 | A | DVS | 4 | 9 | Negative | 0 |
| | | | | | control | |
| 8 | A | DVS | 4 | 8 | 18 | + |
| 9 | A | DVS | 4 | 9 | 18 | |
| 10 | A | DVS | 4 | 10 | 18 | +++ |
| 11 | B | DVS | 4 | 9 | Negative | 0 |
| | | | | | control | |
| 12 | B | DVS | 4 | 8 | 3 | |
| 13 | B | DVS | 4 | 9 | 3 | ++ |
| 14 | B | DVS | 4 | 10 | 3 | +++ |
| 15 | B | DVS | 4 | 9 | Negative | 0 |
| | | | | | control | |
| 16 | B | DVS | 4 | 8 | 18 | + |
| 17 | B | DVS | 4 | 9 | 18 | ++ |
| 18 | B | DVS | 4 | 10 | 18 | +++ |
| 19 | C | DVS | 4 | 9 | Negative | + |
| | | | | | control | |
| 20 | C | DVS | 4 | 8 | 3 | |
| 21 | C | DVS | 4 | 9 | 3 | +++ |
| 21 | C | DVS | 4 | 10 | 3 | ++++ |
| 22 | C | DVS | 4 | 9 | Negative | + |
| | | | | | control | |
| 23 | C | DVS | 4 | 8 | 18 | ++ |
| 24 | C | DVS | 4 | 9 | 18 | +++ |
| 25 | C | DVS | 4 | 10 | 18 | ++++ |

The experiment illustrated the possibility to first activate the dressings and to control the loading of a test protein by coupling at different pH and time. The higher the pH—the more coupled. And the longer the reaction time, the more loading. The effect of pH seemed dominant. Though, the longer coupling time with protein did not give much extra fluorescence. This information was used to optimize the coupling of the tissue factor.

In the following, the coupling of tissue factor to DVS or epichlorohydrin activated dressing is disclosed.

### Example 4—Coupling of Tissue Factor or Mixtures of Tissue Factor and BSA to DVS Activated Dressings at Different pH and Concentrations and Test of Coagulation Efficiency

Tissue Factor (SEQ ID NO: 2) with several glycosylation sites,

(SGTT-
NTVAAYNLTWKSTNFK
TILEWEPKPVNQVYTVQIST
KSGDWKSKCFYTTDTECDLT
DEIVKDVKQTYLARVFSYPA
GNVESTGSAGEPLYENSPEF
TPYLETNLGQPTIQSFEQVG
TKVNVTVEDERTLVRRNNTF
LSLRDVFGKDLIYTLYYWKS
SSSGKKTAKTNTNEFLIDVD
KGENYCFSVQAVIPSRTVNR
KSTDSPVECMGQEKGEFRE)

was obtained from Human Cell Inc. (Naperville, US-IL) and produced in a Cell Growth Production system in EX-CELL293 serum-free medium as described by Sahni, M, Wilcox, S, et al, SAFC Biosciences Research Report called “HEK 293 Cell Growth and Virus Production in EX-CELL™293 Serum-Free Medium, by Manisha Sahni, et. al. Research Report, SAFE Biosciences, www.Safcbiosciences.com issued 2006-R022, 0103. The untagged Tissue Factor was purified by ion exchange and Heparin affinity chromatography before being freeze-dried from a carbonate buffer in 1 mg vials.

A standard solution was prepared by dissolving 1.00 mg Tissue Factor in 0.250 ml deionized water (4.0 mg/ml). The solution was clear, and without any cloudiness.

The coupling was done as described in example 3. Except that some of the conjugations were done with a mixture of Tissue Factor and fluorescein labelled BSA. The table below (Example 5) summarizes the combinations.

The reaction was quenched with the stop buffer for 15 minutes. The dressings were washed extensively by shaking in deionized water three times, followed by 20 mM Tris at pH 7.0 and once with water. The dressings were stored in 10 mm HEPES at pH 7.0, before being tested for the ability to initiate haemostasis.

### Example 5—Ex-Vivo Test of Tissue Factor Functionalized Dressings

The Tissue Factor functionalized dressings (approx. 2×2 cm) and a negative and positive control were placed in small vials and added 5.0 ml fresh human blood. After 20 minutes at room temperature, the dressings were removed from the vial and placed on microscope glasses. The blood clotting and dressing was evaluated by dragging the blood components apart using pipettes. The positive examples all had clear blood clots in the form of dark lumps and or fibre structures. The most positive was gel-like or semisolid state contained continuous and dense lumps. The negative had low viscosity blood without clots. The clotting was scored from ‘−’ to ‘++++’ by two experts.

The following table summarizes the various combinations and the results for Epichlorohydrin activated dressings.

| | | | | Tissue | | | | |
|---|---|---|---|---|---|---|---|---|
| | Internal | Dressing | Activation | Factor | BSA | Coupling | Coupling | Blood |
| No. | Code | type | time | mg/ml | mg/ml | pH | time | clotting |
| 1 | 1B | B | 4 | 0.10 | 0.90 | 9 | 3 | + |
| 2 | 1C | C | 4 | 0.10 | 0.90 | 9 | 3 | +++ |
| 3 | 2B | B | 1 | 0.50 | 0 | 9 | 3 | ++ |
| 3 | 2C | C | 1 | 0.50 | 0 | 9 | 3 | +++ |
| 4 | 3B | B | 1 | 0.10 | 0 | 9 | 3 | + |
| 5 | 3C | C | 1 | 0.10 | 0 | 9 | 3 | − |
| 6 | 4B | B | 1 | +Control | | 9 | 3 | ++++ |
| 7 | 4C | C | 1 | −Control | | 9 | 3 | + |

In conclusion, the experiment illustrated the forming of blood clots by the Tissue Factor coupled dressings.

In the following, coupling of Tissue Factor to reduced DVS activated dressings (1 hour activation), at lower pH and concentrations and test of coagulation efficiency is described.

This experiment is conducted example 4—but with lower pH and concentrations. At pH 8.0, the reaction buffer was 0.10 M HEPES pH 8.0. The blood clotting was evaluated as above.

The following table summarizes the various combinations and the results for the DVS activated dressings:

| | Internal | Dressing | Activation | Tissue Factor | Coupling | Coupling | Blood |
|---|---|---|---|---|---|---|---|
| No. | Code | type | time, hours | mg/ml | pH | time, hours | clotting |
| 6a | 6b | B | 1 | 0.50 | 9 | 1 | + |
| 6 | 6c | C | 1 | 0.50 | 9 | 1 | ++++ |
| | 7b | B | 1 | 0.10 | 9 | 1 | + |
| | 7c | C | 1 | 0.10 | 9 | 1 | + |
| | 8b | B | 1 | 0.02 | 9 | 1 | + |
| | 8c | C | 1 | 0.02 | 9 | 1 | (+) |
| | 9b | B | 1 | 0.10 | 8 | 1 | ++ |
| | 9c | C | 1 | 0.10 | 8 | 1 | ++++ |
| | 10b | B | 1 | 0.02 | 8 | 1 | − |
| | 10c | C | 1 | 0.02 | 8 | 1 | ++ |
| | 11b | B | 4 | −control | 9 | 1 | − |

In summary, the experiment illustrated the ability by Tissue Factor linked to dressing to initiate blood clotting, even when coupling at low Tissue Factor concentrations and at pH 8.0. This could indicate good activity after coupling through thiols on the Tissue Factor protein.

### Example 6—Ex-Vivo Test of Tissue Factor Functionalized Dressings at 37° C. and Covered with Factor VH

A vial of NovoSeven (NovoNordisk, Denmark) was opened and the 1.0 mg freeze dried powder dissolved in 1.00 ml of the supplied buffer solution, to make a Factor VII stock solution.

In the samples covered with Factor VII solution, first excess water was removed from the dressings before they were covered with 1:10 diluted factor VII solution in water.

A new set of tissue factor functionalized dressings was made, based on the vinylsulfon and epichlorohydrin activated “C” dressings, cotton sheets. Coupling for 60 minutes at room temperature, 0.050 mg/ml tissue factor, pH 8.00 in carbonate buffer, quenched with ethanolamine, washed in water and Tris buffer. Only the type of activated dressing and addition of factor VII (“F7”) was varied.

The ex-vivo test was performed as described above, except for an incubation temperature of 37° C. in a heating block and monitoring after 5 minutes, in order to better simulate the real-life situation. All the samples were tested in duplex.

The results are summarized in the table below.

| | Internal | | Blood |
|---|---|---|---|
| No. | Code | Dressing type | clotting |
| 1 | 21a | Not activated | − |
| 2 | 21b | Not Activated | − |
| 3 | 32a | DVS | + |
| 4 | 32b | DVS | + |
| 5 | 42a | Epox | + (+) |
| 6 | 42b | Epox | + (+) |
| 7 | 61a | Not activated + F7 | +++ |
| 8 | 61b | Not activated + F7 | +++ |
| 9 | 71a | DVS + F7 | +++ |
| 10 | 71b | DVS + F7 | +++ |
| 11 | 72a | DVS + F7 | ++ |
| 12 | 72b | DVS + F7 | + (+) |
| 13 | 81a | Epox + F7 | +++ (+) |
| 14 | 81b | Epox + F7 | |
| 15 | 82a | Epox + F7 | +++ |
| 16 | 82b | Epox + F7 | |

In conclusion, the dressings with tissue factor produced clots and the placebo did not.

The dressings covered with Factor VII produced a very dense blood clot, compared to any of the other dressings. We noted that the placebo (not activated) dressing with Factor VII did produce some clear clots, but they were not as dense or rubbery as with the dressings with Tissue Factor. The epichlorohydrin seems better than the DVS dressings in this experiment. We conclude that Factor VII with Tissue Factor linked to dressing enhance blood clotting even more.

### Example 7—Large Scale Preparation of Dressing with Tissue Factor

The activated dressing (type C,) was prepared as in examples 1 and 2.

DVS activated dressing was prepared using 200 ml 5% DVS, pH 10.6 carbonate for 6 pieces of 10×10 cm cotton dressing (approx. 10 gram).

Epichlorohydrin activated dressing was prepared using 200 ml 5% epichlorohydrin, 2 N NaOH in 50% DMSO/water for 6 pieces of 10×10 cm cotton dressing (approx. 10 gram).

The DVS activated dressings shrunk about 5% in length, and the epoxy dressings about 10% in length.

The dressings were cut in 2.5 cm by 2.5 cm pieces and coupled with tissue factor at pH 8 as described above for one hour, with 0.050 mg/ml or 0.100 mg/ml tissue factor, quenched with ethanol amin buffer and washed with water and Tris buffer. Stored in water in the cold. The dressings are summarized in the next table as TL1, TL2, TL3 and TL4.

An untreated cotton dressing was washed with water and Tris buffer and named TL-5, the placebo dressing.

The dressings were stored and transported in water at 2-6° C. and used in mini-pig bleeding tests 3 and 6 days after the preparation.

Mini Pig Bleeding Tests

In the following examples, the test of the tissue factor dressings' ability to induce haemostasis in a spleen lesion and carotid arterial bleeding is disclosed.

The dressings described in the present invention were evaluated and compared with commercially available dressings in vivo, in spleen lesion and carotid arterial bleeding models using mini pigs (Ellegaard Gottingen Minipigs A/S, Dalmose, Denmark).

Following dressings were tested in the in vivo model:

| Code | Dressing type |
|---|---|
| TL-1 | DVS activated dressing C, coupled with 0.05 mg/ml |
| | Tissue Factor for 1 hr at pH 8 (i.e. as described above) |
| TL-3 | Epox activated dressing C, coupled with 0.05 mg/ml |
| | Tissue Factor for 1 hr at pH 8 (i.e. as described above) |
| TL-4 | Epox activated dressing C, coupled with 0.10 mg/ml |
| | Tissue Factor for 1 hr at pH 8 (i.e. as described above) |
| TL-5 | Dressing C treated with H2O, i.e. not activated nor |
| | treated with Tissue Factor |
| Tachosil | Commercially available haemostatic dressing containing |
| | fibrinogen and human thrombin, (Takeda Austria GmbH) |
| QuikClot | Commercially available haemostatic nonwoven gauze |
| | impregnated with kaolin (Z-Medical, provided from |
| | Emergo Europe, The Hague, Netherlands) |

Six male mini pigs, 7-9 months old (15-18 kg) were used in the in vivo studies. Anesthesia was induced by i.m. injection of a zoletil mix (Zoletil 50 (50 mg/ml)+6.25 ml xylazine (20 mg/ml)+1.25 ml Ketamine (100 mg/ml)+2.5 ml butorphanol (10 mg/m)). Dose 1 ml/10 kg i.m. After induction, anesthesia was sustained by isofluran (0-4%).

I.v. catheter (ear vein or femoral vein) was placed in addition to intraarterial blood pressure probe (in femoral arteria). A sample of 4.5 ml in sodium-citrate glass vials was drawn for coagulation measurement by thromboelastographic (TEG 6s, Haemonetics, MA—USA). The pigs were given 25 i.u. heparin/kg i.v. and a new TEG measurement was done after 10 minutes to ensure that the coagulation reached human character, i.e. TEG value that corresponds to the higher end of the human spectrum. Basic volume-therapy was sustained by infusion of ringer-lactate (approximately 5-15 ml/h) in the i.v. catheter.

During the following experiments, the mini pigs were continuously monitored for blood pressure, oxygen saturation and pulse.

### Example 8—Spleen Lesions

A midline incision of 8 cm just below xyphoid in caudal direction was placed. The spleen was localized and placed on the abdomen. The spleen was kept moistened by isotonic NaCl as needed. Spleen lesions were done as 2 sets of 2 standardized scalpel lesions (3 mm deep, and 1.5 cm long), spleen lesions 1 and 2 were made in the distal part and spleen lesion 3 and 4 in the caudal part of the spleen. After 20 seconds of unrestrained bleeding, a dressing 2 cm×2 cm was placed centrally on top of the lesions. Manually compression by a finger was placed for 1 or 3 minutes. After compression, haemostasis was evaluated after approximately 0, 3, 5 or 6 and 10 minutes. After evaluation, the spleen was wrapped in a single layer of gauze with isotonic NaCl and relocated to abdomen.

The dressings tested in the spleen lesion model and the haemostatic effect of the dressings are summarized in the table below:

| | | Spleen | Spleen | Spleen | Spleen |
|---|---|---|---|---|---|
| Object | Trauma | lesion 1 | lesion 2 | lesion 3 | lesion 4 |
| Pig A | Dressing tested | TL-1 | Tachosil | TL-3 | Tachosil |
| | Evaluation | Bleeding | Bleeding | Bleeding | Haemostasis |
| | 0 minutes after | | | | |
| | compression | | | | |
| | Evaluation | Bleeding | Bleeding | Haemostasis | Bleeding |
| | 3 minutes after | | | | |
| | compression | | | | |
| | Evaluation | Haemostasis | Bleeding | Haemostasis | Haemostasis |
| | 6 minutes after | | | | |
| | compression | | | | |
| | Evaluation | Haemostasis | Bleeding | Haemostasis | Haemostasis |
| | 10 minutes after | | | | |
| | compression | | | | |
| Pig B | Dressing tested | TL-3 | TL-5 | TL-5 | TL-3 |
| | Evaluation | Haemostasis | Bleeding | Bleeding | Bleeding |
| | 0 minutes after | | | | |
| | compression | | | | |
| | Evaluation | Haemostasis | Slight | Haemostasis | Haemostasis |
| | 3 minutes after | | bleeding | | |
| | compression | | | | |
| | Evaluation | Haemostasis | Haemostasis | Haemostasis | Haemostasis |
| | 6 minutes after | | | | |
| | compression | | | | |
| | Evaluation | Haemostasis | Haemostasis | Haemostasis | Haemostasis |
| | 10 minutes after | | | | |
| | compression | | | | |
| Pig C | Dressing tested | TL-5 | TL-1 | TL-5 | TL-1 |
| | Evaluation | Bleeding | Bleeding | Haemostasis | Haemostasis |
| | 0 minutes after | | | | |
| | compression | | | | |
| | Evaluation | Haemostasis | Bleeding | Slight | Haemostasis |
| | 3 minutes after | | | bleeding | |
| | compression | | | | |
| | Evaluation | Slight | Bleeding | Haemostasis | Haemostasis |
| | 6 minutes after | bleeding | | | |
| | compression | | | | |
| | Evaluation | Haemostasis | Haemostasis | Haemostasis | Haemostasis |
| | 10 minutes after | | | | |
| | compression | | | | |
| Pig D | Dressing tested | TL-3 | TL-5 | TL-3 | TL-5 |
| | Evaluation | Bleeding | Bleeding | Bleeding | Bleeding |
| | 0 minutes after | | | | |
| | compression | | | | |
| | Evaluation | Haemostasis | Haemostasis | Haemostasis | Bleeding |
| | 3 minutes after | | | | |
| | compression | | | | |
| | Evaluation | Haemostasis | Haemostasis | Haemostasis | Bleeding |
| | 5 minutes after | | | | |
| | compression | | | | |
| | Evaluation | Haemostasis | Haemostasis | Haemostasis | Haemostasis |
| | 10 minutes after | | | | |
| | compression | | | | |
| Pig E | Dressing tested | TL-5 | TL-4 | TL-4 | TL-5 |
| | Evaluation | Bleeding | Bleeding | Bleeding | Bleeding |
| | 0 minutes after | | | | |
| | compression | | | | |
| | Evaluation | Bleeding | Bleeding | Slight | Slight |
| | 3 minutes after | | | bleeding | bleeding |
| | compression | | | | |
| | Evaluation | Bleeding | Bleeding | Haemostasis | Haemostasis |
| | 5 minutes after | | | | |
| | compression | | | | |
| | Evaluation | Haemostasis | Haemostasis | Haemostasis | Haemostasis |
| | 10 minutes after | | | | |
| | compression | | | | |
| Pig F | Dressing tested | Tachosil | TL-3 | TL-3 | TL-5 |
| | Evaluation | Bleeding | Bleeding | Bleeding | Bleeding |
| | 0 minutes after | | | | |
| | compression | | | | |
| | Evaluation | Haemostasis | Haemostasis | Haemostasis | Haemostasis |
| | 3 minutes after | | | | |
| | compression | | | | |
| | Evaluation | Bleeding | Haemostasis | Haemostasis | Haemostasis |
| | 5 minutes after | | | | |
| | compression | | | | |
| | Evaluation | Haemostasis | Haemostasis | Haemostasis | Haemostasis |
| | 10 minutes after | | | | |
| | compression | | | | |

The experiment illustrates the ability of the Tissue Factor coupled dressings to induce haemostasis in the spleen lesion model. Compared to Tachosil or placebo (TL-5), the tissue factor dressings induced haemostasis at the same time or faster.

In general, For Tachosil, bleeding started again after initial haemostasis. Tissue Factor dressings formed a dense blood clot, which effectively stopped the bleeding and made a mechanically stable blockage. It was noted that the Tissue Factor dressings seem to introduce thickening of the running blood, whereas blood running from the Tachosil dressing remained liquid.

### Example 9—Carotid Arterial Bleeding Model

After the spleen model experiment was finalized, the ringer-lactate infusion velocity was increased to approximately 50 ml/hr as preparation for the arterial lesion model. Henceforward, adjustment of the infusion velocity was based on the blood pressure (BP) aiming to keep a mean BP above 65 mmHg during the study. The carotid artery was exposed and a guiding line placed at top and bottom in order to be able to lift the arteria for incision and bleeding control during the study. A 1-3 ml lidocaine 20% on gauze was placed directly on the artery. During lifting, the blood-flow in the artery was obstructed and a lesion by puncture with a 14G cannula was induced. Relaxing of the lifting of the artery showed arterial bleeding. If the bleeding was not pulsative, the lesion size was increased. 20 seconds after the arteriotomy and unrestrained bleeding, two layers of dressing were placed on the lesion, with 2 layers of gauze on top followed by manual compression. After 5 minutes, the manual compression was slowly displaced and the site was examined for bleeding. If no bleeding was seen, the gauze was carefully removed and the dressing was left untouched for approximately 3-5 minutes. If haemostasis was not obtained, another dressing (two layers) was placed on the bleeding followed by 5 minutes of manual compression. During the study, BP was closely monitored.

The study was ended by injection of pentobarbital i.v.

Results obtained in the carotid arterial bleeding model is summarized below:

| | | Carotid arterial lesion by puncture with a 14G cannula | | |
|---|---|---|---|---|
| | Trauma | | Second | Third |
| Object | Treatment | First treatment | treatment | treatment |
| Pig A | Dressing tested | TL-4 | Not Relevant | |
| | Evaluation 0 minutes | Haemostasis | | |
| | after compression | | | |
| | Evaluation 5 minutes | Haemostasis | | |
| | after compression | | | |
| Pig B | Treatment | TL-5 (two layers) | TL-3 (two layers | Not Relevant |
| | | Placebo | | |
| | Evaluation 0 minutes | Excessive | Haemostasis | |
| | after compression | bleeding | | |
| | Evaluation 3 minutes | | Haemostasis | |
| | after compression | | | |
| Pig C | Treatment | QuickClot | TL-5 (two layers) | TL-4 (two layers) |
| | | (Z-Medical) | Placebo | |
| | Evaluation 0 minutes | Excessive | Excessive | Haemostasis |
| | after compression | bleeding | bleeding | |
| | Evaluation 3 minutes | | | Haemostasis |
| | after compression | | | |
| Pig D | Treatment | TL-3 | Not Relevant | |
| | Evaluation 0 minutes | Haemostasis | | |
| | after compression | | | |
| | Evaluation 5 minutes | Haemostasis | | |
| | after compression | | | |
| Pig E | Treatment | QuickClot | TL-3 | Not Relevant |
| | Evaluation 0 minutes | Excessive | Haemostasis | |
| | after compression | bleeding | | |
| | Evaluation 5 minutes | | Haemostasis | |
| | after compression | | | |

| | | | |
|---|---|---|---|
| Pig F | Treatment | QuickClot | Not Relevant |
| | Evaluation 0 minutes | Haemostasis | |
| | after compression | | |
| | Evaluation 5 minutes | Hemostasis | |
| | after compression | | |

Results obtained in the carotid arterial bleeding model show that the Tissue Factor coupled dressings tested are able to stop excessive arterial bleedings.

The QuikClot dressing worked in one of the examples but failed in the other examples. The Tissue Factor dressings performed efficiently in all the examples—even after QuikClot and placebo failed, as illustrated for mini pig C, where also blood pressure was restored after compression and haemostasis with TL-3.

### Example 10—Stability of Tissue Factor and Tissue Factor Coupled Dressings

Dressings from example 7 (3 pieces, approx. 3×3 cm) and used in the mini pig bleeding experiments, examples 8 and 9, were stored in the dark for 10 months in 500 ml filtered MilliQ water at 4-7° C. without any preservatives. Round pieces were punched out and the ability to coagulate plasma was tested in a standardized ex-vivo assay setup:

In more detail, dressing pieces were punched out from the prepared dressings. Punched dressings (diameter 4 mm or 5 mm) were placed in 1.5 ml polypropylene low binding vials (Corning) and added cold 340 ul blood plasma pooled from 5 subjects, 20 ul CaCl₂ and 40 ul buffer A (1% BSA). The vials were placed in a heating block at 37.0° C. Each vial was visually inspected with regular intervals (30-60 sec.): The vials were manually turned upside down and if the mixture stayed in the bottom of the vial, the sample was interpreted as being clotted. The time from added blood plasma to clot was recorded. All measurements were done in doublets.

A standard dilution series in buffer A of freshly prepared Tissue Factor from HEK293, (SEQ ID NO: 2), same as in example 4, was used for reference.

The results are summarized in the table below.

| | | Clotting | Clotting |
|---|---|---|---|
| | | time | time |
| | | Vial | Vial |
| | | assay | assay |
| Sample | Amount | sec. (a) | sec. (b) |
| TL-3 dressing, 10 Mo. | 3 × 4 mm | 350 | 300 |
| TL-3 dressing, 10 Mo. | 2 × 4 mm | 360 | 360 |
| TL-3 dressing, 10 Mo. | 1 × 4 mm | 435 | 435 |
| TL-1 dressing, 10 Mo. | 3 × 4 mm | 300 | 345 |
| TL-2 dressing, 10 Mo. | 3 × 4 mm | 345 | 345 |
| TL-4 dressing, 10 Mo. | 3 × 4 mm | 345 | 345 |
| C dressing unmodified | 2 × 4 mm | 540 | 540 |
| Reference, water | 40 uL | 1080 | 1080 |
| Free TF | 0.010 mg/mL | 488 | 488 |
| Free TF | 0.002 mg/mL | 602 | 664 |
| Free TF | 0.0004 mg/mL | 953 | 953 |
| Free TF | 0.00008 mg/mL | 880 | 880 |

In conclusion, the 10-month-old TL3 dressing could still initiate clotting of the blood plasma. The same dressing was used in the mini pig experiment in example 8 and 9.

TL4 with a different conjugation ratio could still initiate clotting. A similar dressing, TL1 and TL2, made with DVS activation and the different conjugation ratios, could also initiate clotting.

Also, the Tissue Factor seems not to have leaked out into the large volume liquid after months of storage.

The results further illustrate a dose-response effect, as 3 pieces of TL3 dressing gave a shorter clotting time as 1 piece.

The clotting efficiency of 3 round pieces of TL3 of 4 mm in diameter is better than approximately 0.010 mg/ml free Tissue Factor, as judged from the Tissue Factor dilution series, although the values cannot be directly compared because the Tissue Factor in the dressing material is likely more dense than the equivalent amount in free solution.

### Example 11—Reproducibility and Leakage from Dressings

In this example, the reproducibility of preparing fresh Tissue Factor dressings with no leakage of Tissue Factor was explored further. QuikClot dressing was included for comparison.

Epichlorohydrin activated dressing C, coupled with 0.05 mg/ml Tissue Factor for 1 hr. at pH 8 (prepared as TL-3 in example 7 above) was after the quenching step with buffered ethanolamine (10 ml) washed with 10 ml water 7 times. Both the dressing and wash liquid were analysed for the ability to coagulate blood plasma in the vial assay in doublets (a and b), as described in example 10 above. The clotting time was recorded.

20 round pieces of 4 mm in diameter QuikClot dressing (Z-Medical, impregnated with kaolin) was sequentially washed for comparison (100 ul water per 4 mm round piece). In especially the first wash cycle and all the following wash cycles, a white precipitate was visible in the wash liquid. 6 dressing pieces were removed for test after 1, 3 and 7 washes and wash liquid collected. The samples were tested in the manual vial assay. Table below summarizes the experiments:

| | | Clotting | Clotting |
|---|---|---|---|
| | | time | time |
| | | Vial | Vial |
| | | assay | assay |
| Sample | Amount | sec. (a) | sec. (b) |
| New TL-3 dressing | 3 × 5 mm | 390 | 390 |
| New TL-3 dressing | 2 × 5 mm | 450 | 450 |
| New TL-3 dressing | 1 × 5 mm | 480 | 480 |
| Wash liquid after 7 washes | 40 uL | More | More |
| | | than | than |
| | | 1200 | 1200 |
| QuikClot dressing, not washed | 3 × 4 mm | 240 | 240 |
| QuikClot after wash no. 1 | 3 × 4 mm | 300 | 300 |
| QuikClot after wash no. 3 | 3 × 4 mm | 392 | 345 |
| QuikClot after wash no. 7 | 3 × 4 mm | 392 | 345 |
| QuikClot Wash liquid no. 1 | 40 uL | 300 | 300 |
| QuikClot Wash liquid no. 3 | 40 uL | 420 | 420 |
| QuikClot Wash liquid no. 7 | 40 uL | 960 | 900 |
| Reference, water | 40 uL | 1140 | 900 |

In conclusion, the Tissue Factor activated dressing can be washed free of unbound Tissue Factor after conjugation. After 7 wash circles, the wash liquid cannot initiate clotting, whereas the bound Tissue Factor on the dressing could initiate clotting.

The results again illustrate a dose-response effect, as 3 pieces of new TL-3 dressing give a shorter clotting time as 1 piece.

In comparison, QuikClot's kaolin could be partly washed out and the wash liquid was able to initiate clotting even after 7 wash circles. The washed QuikClot could still initiate clotting. It is noted, that the two different types of dressing initiate with different mechanisms.

### Example 12—Dressing with Tissue Factor from E. coli

Non-glycosylated recombinant Tissue Factor (SEQ ID NO: 2) (see amino acid sequence in example 4 above) were expressed from E. coli and obtained from Elab-science Biotechnology Inc. (Wuhan, China and Houston, US-Tx) In more detail, using pET-28a vector, expressed with N-terminal fused with GST tag, affinity purified, Tag cleaved and SEC and ion exchange purified. Shipped lyophilized in vials of 0.10 mg).

Standard dilution series in Buffer A (BSA, PBS) of freshly prepared Tissue factor from E. coli and HEK293 was compared in a standard ex-vivo TEG® 5000 Haemostasis Analyzer System (Thromboelastography, Haemonetics Corporation).

Clotting time from the enzymatic reaction of coagulation factors alone was recorded as the R value. The clot kinetics parameters were also recorded.

In short, 40 ul samples in Buffer A to be measured were added 20 ul CaCl2 340 ul blood plasma pooled from 5 subjects in the measuring cup in the TEG instrument. The R value was recorded as the clotting time from the coagulation factors alone. All measurements were done in doublet experiments.

A dressing with coupled Tissue Factor from E. coli was prepared as described in example 7 and tested in the vial assay.

Also, a freshly prepared dressing from example 11, with Tissue Factor from HEK293 and an unmodified dressing was included.

The results are summarized in the Table below.

| | | | | Vial | Vial |
|---|---|---|---|---|---|
| | | TEG, | TEG, | assay | assay |
| | Conc. | R, sec. | R, sec. | sec. | sec. |
| Sample | mg/ml | (a) | (b) | (a) | (b) |
| Free TF (e-coli) | 0.010 | 402 | 660 | 900 | 800 |
| Free TF (e-coli) | 0.002 | 774 | 1044 | 1148 | 1057 |
| Free TF (e-coli) | 0.0004 | 792 | 1032 | 1002 | 1208 |
| Free TF (e-coli) | 0.00008 | 1362 | 1242 | 1211 | 1067 |
| Water, ref | | | | 1448 | 1221 |
| Free TF (HEK) | 0.010 | 330 | 324 | 300 | 300 |
| Free TF (HEK) | 0.002 | 486 | 468 | 345 | 345 |
| Free TF (HEK) | 0.0004 | 612 | 540 | 525 | 525 |
| Free TF (HEK) | 0.00008 | 942 | 954 | 300 | 300 |
| Water, ref | 0 | | | 1090 | More |
| | | | | | than |
| | | | | | 1200 |
| FT (e-coli) coupled | 3 × 5 mm | | | 850 | 725 |
| to Dressing type C | diameter | | | | |
| FT (HEK) coupled | 3 × 5 mm | | | 390 | 390 |
| to Dressing type C | diameter | | | | |
| Reference-not | 3 × 5 mm | | | 900 | More |
| modified dressing | diameter | | | | than |
| | | | | | 1200 |

In conclusion, the free TF from E. coli was able to initiate clotting of the blood plasma, but apparently with a somewhat lower efficiency than the TF from HEK293. The dressing coupled with TF from E. coli could initiate clotting slightly better than the reference dressing. With the same conjugation conditions, as for the Tissue Factor from HEK cells, the clotting time for the Tissue Factor from E. coli dressing was longer.

1. A system for promoting rapid haemostasis while reducing the risk of undesirable blood coagulation, said system comprising:
Tissue Factor (TF) or any variant thereof,
a dressing material to which the Tissue Factor or any variant thereof is linked in such a way that the Tissue Factor or any variant thereof is prevented from dissociating from the dressing material when exposed to a physiological environment.

2. The system of claim 1, wherein the Tissue Factor or any variant thereof is of human, porcine, equine, canine or bovine origin.

3. The system of claim 1, wherein the Tissue Factor or any variant thereof is human Tissue Factor.

4. The system according to claim 1, wherein the Tissue Factor is the extracellular domain of human Tissue Factor.

5. The system according to claim 1, wherein said Tissue Factor comprises SEQ ID NO: 2 or any variant thereof having at least 60%, such as at least 70%, at least 80%, at least 90%, or at least 95% sequence identity with SEQ ID NO: 2.

6. The system according to claim 1, wherein said Tissue Factor is recombinant human Tissue Factor having the SEQ ID NO: 2.

7. The system according to claim 1, wherein the dressing material is selected from the group consisting of wound dressings, patches, gauzes, extracellular matrix components, gels, creams, ointments, particles, such as nanoparticles, and any combination thereof.

8. The system according to claim 1, wherein the dressing material is selected from the group consisting of cotton, cellulose, cellulose ethers, regenerated cellulose, oxidized cellulose, carboxymethylcellulose, polyurethane, agarose or paper based membranes, polyamides, poly sulfone ether, polyvinyl alcohols, nitrocellulose, nitrocellulose mixed esters, nylons, polycarbonate, polysulfone, polyethylene terephthalate, polyvinylidene fluoride or polypropylene, polyethylene and co-block polymers, blends and combinations thereof.

9. The system of claim 7, wherein the Tissue Factor or any variant thereof is linked to the dressing material by a linker selected from the group consisting of azlactones, epoxides, cyanobromide, N-Hydroxysuccinimide esters, nitrophenyl esters and other active esters, aryl halides, isothiocyanates, aldehydes, maleimide, disulphides, cyclic lactones, triazines, benzoequinone, and vinylsulfones.

10. The system of claim 1, wherein the linkage is based on covalent bonding.

11. The system of claim 1, wherein the dressing material is made from a material, which is absorbable and/or non-absorbable in mammalian plasma/blood.

12. The system of claim 1 further comprising Factor VII/VIIa.

13. The system of claim 1 further comprising a Tissue Factor stabilizing agent.

14. The system of claim 13, wherein the Tissue Factor stabilizing agent is a protein selected from the group comprising serum albumin, gelatin, casein and derivatives thereof.

15. The system of claim 13, wherein the Tissue Factor stabilizing agent is attached to the dressing material.


SEQUENCE LISTING (4 sequences)

SEQ ID NO: 1 - DNA, 663 bp, Homo sapiens
  extracellular domain of human Tissue Factor (1)..(663)
tcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaatgataa

SEQ ID NO: 2 - protein, 219 aa, Homo sapiens
  extracellular domain of human Tissue Factor (1)..(219)
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 3 - protein, 71 aa, Homo sapiens
  Conserved sequence Tissue Factor (1)..(71)
AYNLTWKSTNFKTILEWEPKPVNQVYTVQISTKSGDWKSKCFYTTDTECDLTDEIVKDVKQTYLARVFSYP

SEQ ID NO: 4 - protein, 124 aa, Homo sapiens
  Tissue Factor conserved sequence (1)..(124)
EPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEK